(19) United States
(12) Patent Application Publication
(10) Pub. No.: US 2026/0345376 A1
(43) Pub. Date: Nov. 5, 2026

(54) METHOD OF TREATING OR PREVENTING ALOPECIA USING CRYOTHERAPY AND CRYOTHERAPY APPARATUS THEREFOR

(71) Applicant: RECENSMEDICAL, INC., Hwaseong-si Gyeonggi-do (KR)

(72) Inventors: Gun-Ho KIM, Hwaseong-si Gyeonggi-do (KR); Ohsang KWON, Seoul (KR); Hyeongyeong KIM, Seoul (KR); Seunghee LEE, Seoul (KR)

(73) Assignee: RECENSMEDICAL, INC., Hwaseong-si Gyeonggi-do (KR)

(21) Appl. No.: 19/654,842

(22) Filed: Apr. 22, 2026

(30) Foreign Application Priority Data
Apr. 30, 2025 (KR) 10-2025-0056911

(51) Int. Cl. A61F 7/00

(52) U.S. Cl. CPC A61F 7/0085; A61F 2007/0008; A61F 2007/0052; A61F 2007/0063; A61F 2007/0086; A61F 2007/0087; A61F 2007/0093; A61F 2007/0095

(57) ABSTRACT

The present disclosure discloses a cryotherapy apparatus of treating or preventing androgenetic alopecia of a human. Especially, the cryotherapy apparatus comprises: a valve configured to allow a flow of a coolant which is provided from a coolant storage; a temperature regulator configured to regulate a temperature of the coolant; a nozzle configured to spray the coolant; a temperature sensor configured to measure a temperature of a target area which is a part of a human scalp; and a controller configured to the valve, the temperature sensor and the temperature regulator.

### CROSS REFERENCE TO RELATED APPLICATION

The present application claims priority to Korean Patent Application No. 10-2025-0056911, filed Apr. 30, 2025, the entire contents of which is incorporated herein for all purposes by this reference.

### BACKGROUND

### 1. Technical Field

The present disclosure relates to a method of treating or preventing alopecia using cryotherapy and a cryotherapy apparatus therefor. More particularly, the present disclosure relates to a method of treating or preventing alopecia by cooling the temperature of the skin surface to a specific temperature for a specific time and to a cryotherapy apparatus for use in the treatment or prevention of alopecia.

### 2. Description of the Related Art

Alopecia refers to progressive hair loss from body parts where hair should exist. In particular, alopecia typically refers to hair loss from the scalp. More precisely, alopecia refers to a condition in which the number of hairs falling out exceeds the number of hairs growing from the scalp, resulting in areas where hair is gradually absent are occurred. When alopecia occurs, this leads to an unattractive appearance from an aesthetic perspective. Accordingly, individuals experiencing alopecia suffer from poor self-confidence regarding their appearance, which is problematic. In severe cases, alopecia may lead to depression. In addition, with the increasing interest in beauty and appearance in modern society, there is a growing demand for alopecia treatment. Accordingly, the market for alopecia treatment is also gradually increasing in scale.

In the meantime, although scalp cooling is known to be effective in preventing and treating alopecia, specific cooling conditions, such as cooling time and cooling temperature of the scalp, have remained undetermined. Accordingly, alopecia treatment using cryotherapy inevitably relies on the practitioner's experience, There was a problem in that the reliability of the effect was reduced.

### DOCUMENT OF RELATED ART

### Non-Patent Document

Medical News Internet Article (Title: “Cooling Caps”Proven Effective for Alopecia in Cancer Patients . . . ” “Ongoing Efforts to Expand Their Use”/Publication Date: Mar. 15, 2024/URL address: http://www.bosa.co.kr/news/articleView.html?idxno=2218555)

### SUMMARY

The present disclosure aims to describe a treatment method for treating and preventing alopecia using cryotherapy.

Specifically, the present disclosure aims to provide a cooling temperature and cooling time for treating and preventing alopecia. In addition, the present disclosure aims to provide an administration method for treating and preventing alopecia.

Technical problems to be solved by the present disclosure are not limited to the aforementioned technical problems, and other technical problems which are not mentioned will be clearly understood by those skilled in the art from the present disclosure.

According to the present disclosure, a cryotherapy apparatus of treating or preventing androgenetic alopecia of a human may comprise: a valve configured to allow a flow of a coolant which is provided from a coolant storage; a temperature regulator configured to regulate a temperature of the coolant; a nozzle configured to spray the coolant; a temperature sensor configured to measure a temperature of a target area which is a part of a human scalp; and a controller configured to the valve, the temperature sensor and the temperature regulator. wherein the controller is further configured to: set a target temperature to cool and maintain the target area to the set target temperature, receive, from the temperature sensor, the measured temperature of the target area, control the temperature regulator to regulate a temperature of the coolant based on the target temperature and the measured temperature of the target area, and control the valve to spray the coolant through the nozzle such that the temperature of the target area reaches to the target temperature and a cooling time of the target area is equal to or longer than a total cooling time period, in characterized that the target temperature may be equal to or less than 5 degrees Celsius and the total cooling time period is equal to or longer than 17 seconds.

Furthermore, according to the present disclosure, a cryotherapy apparatus of treating or preventing alopecia of a human may comprise a valve configured to allow a flow of a coolant which is provided from a coolant storage; a nozzle configured to spray the coolant; and a controller configured to the valve, wherein the controller is further configured to: set a target temperature to cool and maintain a target area, which is a part of a human scalp, to the set target temperature, and control the valve to spray the coolant through the nozzle such that a temperature of the target area reaches to the target temperature and a cooling time of the target area is equal to or longer than a total cooling time period, in characterized that the target temperature may be equal to or less than 5 degrees Celsius and the total cooling time period is equal to or longer than 17 seconds.

According to one embodiment of the present disclosure, alopecia in an animal is treatable or preventable.

According to one embodiment of the present disclosure, genetic factors known to treat or prevent alopecia in an animal can be activated.

According to one embodiment of the present disclosure, the anagen phase of hair growth in an animal can be induced.

Technical problems to be solved by the present disclosure are not limited to the aforementioned technical problems, and other technical problems which are not mentioned will be clearly understood by those skilled in the art from the present disclosure.

### BRIEF DESCRIPTION OF THE DRAWINGS

FIG. 1 shows one example of using a spray-type cryotherapy apparatus to treat or prevent alopecia in an animal, according to one embodiment of the present disclosure;

FIGS. 2 and 3 are views illustrating spray-type cooling systems according to embodiments of the present disclosure;

FIGS. 4 and 5 are views illustrating configurations of spray-type cryotherapy apparatuses according to embodiments of the present disclosure;

FIGS. 6 and 7 are views for describing a treatment method or a prevention method using a spray-type cryotherapy apparatus to treat or prevent alopecia, according to embodiments of the present disclosure;

FIG. 8 is a view for describing the operation of a spray-type cryotherapy apparatus to treat or prevent alopecia, according to one embodiment of the present disclosure;

FIG. 9 shows one example of using a spray-type cryotherapy apparatus to treat or prevent alopecia in a human, according to one embodiment of the present disclosure;

FIGS. 10A and 10B show examples of using a contact-type cryotherapy apparatus to treat or prevent alopecia in an animal or human, according to one embodiment of the present disclosure;

FIG. 11 illustrates configurations of a contact-type cryotherapy apparatus according to one embodiment of the present disclosure;

FIGS. 12 and 13 are views for describing a treatment method or a prevention method using a contact-type cryotherapy apparatus to treat or prevent alopecia, according to embodiments of the present disclosure;

FIG. 14 is a view for describing the operation of a contact-type cryotherapy apparatus to treat or prevent alopecia, according to one embodiment of the present disclosure;

FIG. 15 is a view for describing a treatment method or a prevention method using a spray-type cryotherapy apparatus or a contact-type cryotherapy apparatus to treat or prevent alopecia, according to embodiments of the present disclosure;

FIG. 16 is a view for describing pilot experiments to select experimental groups for experiments on alopecia treatment effects;

FIGS. 17 to 20 are views for describing nonclinical experimental results of experiments on alopecia treatment effects; and

FIGS. 21, 22, 23, 24A, 24B, 25A and 25B are views for describing clinical experimental results of experiments on alopecia treatment effects.

### DETAILED DESCRIPTION

The foregoing objectives, features, and advantages will become more apparent from the following detailed description taken in conjunction with the accompanying drawings. However, the present disclosure may have various changes and various embodiments, and hereinafter, specific embodiments will be illustrated in the drawings and described in detail.

Since the embodiments described in this specification are intended to clearly explain the idea of the present disclosure to those skilled in the art in the field to which the present disclosure pertains, the present disclosure is not limited to the embodiments described herein, but the scope of the present disclosure should be construed to include modifications or variations that do not depart from the spirit of the present disclosure.

The drawings attached to this specification are intended to easily explain the present disclosure. Since the shapes shown in the drawings may be exaggerated as necessary to aid understanding of the present disclosure, the present disclosure is not limited by the drawings.

When it is determined that the detailed description of the known configuration or function related to the present disclosure in this specification may obscure the gist of the present disclosure, detailed explanations regarding this will be omitted as necessary. In addition, ordinals (for example, first, second, and so on) used in this specification are merely used to distinguish one component from another component.

In addition, in the following description, the expressions “unit”, “module”, and “part” contained in terms of constituent elements to be described will be selected or used together in consideration only of the convenience of writing the following specification, and the expressions “unit”, “module”, and “part” do not necessarily have different meanings or roles.

In the following embodiments, singular expressions include plural expressions unless the context clearly indicates otherwise.

In the following embodiments, it will be further understood that the terms “include”, “have”, and so on are intended to indicate the existence of the features or components disclosed in the specification, and are not intended to preclude the possibility that one or more other features or components may exist or may be added.

In the drawings, the size of each component may be exaggerated or reduced for convenience of description. For example, the size and thickness of each component shown in the drawings are arbitrarily indicated for convenience of description, and the present disclosure is not necessarily limited thereto.

In cases in which a certain embodiment may be realized differently, a specific process sequence may be different from the described sequence. For example, two processes described in succession may be performed substantially simultaneously, or may be performed in an order opposite to the described order.

In the following embodiments, when it is said that a film, a region, and a component are connected to each other, it includes not only a case in which the film, the region, and the component are directly connected to each other, but also a case in which other film, region, and component are placed between the film, the region, and the component such that the film, the region, and the component are indirectly connected to each other.

For example, in the present specification, when it is said that a film, a region, and a component are electrically connected to each other, it includes not only a case in which the film, region, and component are directly electrically connected to each other, but also other films, regions, and components are placed between the film, region, and component such that the film, region, and component are indirectly electrically connected to each other.

In the following embodiments, meaning that the film, region, component, and so on are in fluid communication with each other may be interpreted as meaning that each of the film, region, component, and so on forms at least a part of a flow path through which a fluid flows.

For example, in this specification, that a component A is in fluid communication with a component B means that a fluid passing through a flow path formed in the component A can reach a flow path formed in the component B or vice versa. Specifically, when the component A and the component B are coupled to each other and the flow path formed in the component A and the flow path formed in the component B are directly connected to each other, the component A and the component B may be considered to be in fluid communication with each other. Alternatively, when the component A and the component B are connected to each other through a component C, such as a conduit and the flow path formed in the component A and the flow path formed in the component B are indirectly connected to each other through a flow path formed in the component C, the component A and the component B may be considered to be in fluid communication with each other. In this case, it may be interpreted that the component C places the component A in fluid communication with the component B. In addition, the component A and the component B may be in fluid communication with each other through a plurality of components.

A precision cooling technology described herein refers to a technology for precisely adjusting the temperature of a target area, such as a wounded part or the skin, by cooling the target area. Specifically, the precision cooling technology may refer to a technology for cooling the target area until the temperature of the target area reaches a target temperature and maintaining the temperature of the target area at the target temperature for a cooling time.

Maintaining the temperature of the target area at the target temperature for the cooling time may mean adjusting the temperature of the target area to fall within a threshold value preset from the target temperature for the cooling time.

In this case, the threshold value may be selected from 1 °C to 10 °C. For example, when selecting a target temperature of 0 °C, a cooling time of 5 seconds, and a threshold value of 5 °C, a treatment method using the precision cooling technology may refer to a process of maintaining the temperature of the target area at −5 °C or more and 5 °C or less for 5 seconds.

Alternatively, the threshold value is selected from 1 °C to 10 °C, but may be understood as a standard deviation value. For example, when selecting a target temperature of −5 °C, a cooling time of 10 seconds, and a threshold value of 5 °C, the treatment method using the precision cooling technology may refer to a process of maintaining the temperature of the target area such that the standard deviation is 5 °C based on a target temperature of −5 °C for 10 seconds.

Alternatively, the threshold value may be understood as a temperature change rate over time. Specifically, the threshold value may be selected from 1 °C/sec to 30 °C/sec. For example, when selecting a target temperature of 5 °C, a cooling time of 20 seconds, and a threshold value of 5 °C/sec, the treatment method using the precision cooling technology may refer to a process of regulating the instantaneous temperature change rate of the target area to remain below 5 °C/sec.

The target area may refer to an area where a cryotherapy method is performed. For example, the target area may refer to an area where a coolant is sprayed in a cryotherapy method using a spray-type cryotherapy apparatus, to be described later. For another example, the target area may refer to an area in contact with a cooling medium in a cryotherapy method using a contact-type cryotherapy apparatus, to be described later. The size of the target area may be selected depending on the design of the cryotherapy apparatus for performing the cryotherapy method. For example, the size of the target area may be selected as a diameter from 10 mm to 100 mm or as an area from 50 mm² to 100 cm².

The target temperature may be selected from a variety of temperature ranges. For example, the target temperature may be selected from about −20 °C or more and about 30 °C or less. Alternatively, the target temperature may be selected from about −15 °C or more and about 30 °C or less. Alternatively, the target temperature may be selected from about −10 °C or more and about 30 °C or less. Alternatively, the target temperature may be selected from about −5 °C or more and about 30 °C or less. Alternatively, the target temperature may be selected from about −5 °C or more and about 20 °C or less. Alternatively, the target temperature may be selected from about −5 °C or more and about 10 °C or less, about- 10 °C or more and about 5 °C or less, or about −5 °C or more and about 5 °C or less.

The cooling time may be selected from a variety of time ranges. For example, the cooling time may be selected from the range of about 1 second or more and about 300 seconds or less. Alternatively, the cooling time may be selected from the range of about 5 seconds or more and about 300 seconds or less. Alternatively, the cooling time may be selected from the range of about 10 seconds or more and about 300 seconds or less. Alternatively, the cooling time may be selected from the range of about 20 seconds or more and about 300 seconds or less.

According to an Embodiment of the present disclosure, a method of treating or preventing androgenetic alopecia of a human using a cryotherapy apparatus in a subject may comprise: providing the cryotherapy apparatus, wherein the cryotherapy apparatus comprises a nozzle, a valve, a coolant storage connector coupled to a coolant storage, and at least one pipe fluidically connecting the nozzle, the value, and the coolant storage connector; positioning the nozzle of the cryotherapy apparatus to face a target area of the subject, the target area is a part of a human scalp; and operating the cryotherapy apparatus to spray a coolant to the target area such that a temperature of the target area reaches to a target temperature and a cooling time of the target area is equal to or longer than a total cooling time period; in characterized that the target temperature is equal to or less than 5 degrees Celsius and the total cooling time period is equal to or longer than 17 seconds.

Furthermore, the operating the cryotherapy apparatus may comprise operating the cryotherapy apparatus to spray the coolant continuously from a cooling starting time for the total cooling time period to a cooling terminating time for the total cooling time period, and whereby the target area may be cooled to the target temperature and maintained its temperature for the total cooling time period.

Furthermore, the operating the cryotherapy apparatus may comprise operating the cryotherapy apparatus to spray the coolant intermittently from a cooling starting time for the total cooling time period to a cooling terminating time for the total cooling time period, whereby the target area may be cooled to the target temperature and maintained its temperature for a first cooling time period, and then the target area may be cooled again to the target temperature and maintained its temperature for a second cooling time period, wherein a sum of the first cooling time period and the second cooling time period may be same to the total cooling time period.

Furthermore, a cooling pause time period between the first time and the second time may be shorter than at least one of the first cooling time period and the second cooling time period.

Furthermore, the first cooling time period may be substantially identical with the second cooling time period.

Furthermore, the cooling pause time period may be predetermined such that cooling effect of the target area due to the first cooling time period does not disappear during the cooling pause time period.

Furthermore, each of the first cooling time period and the second cooling time period may be equal to or longer than 10 seconds.

Furthermore, the cooling pause time period may be equal to or shorter than 4 seconds.

Furthermore, the total cooling time period may be equal to or shorter than 40 seconds, 42 seconds, 44 seconds, 46 seconds, 48 seconds, or 50 seconds.

Furthermore, the total cooling time period may be equal to or shorter than 60 seconds, 63 seconds, 66 seconds, 69 seconds, 72 seconds, or 75 seconds.

Furthermore, the method may activate at least one of FGF21, VEGF, CIRP, RBM3 and PGC 1a of the human.

Furthermore, the method may induce an anagen phase of hair growth of the human scalp.

Furthermore, the coolant may be liquified carbon dioxide (CO2).

Furthermore, the method may be repeatedly performed for at least 4 weeks, at least 8 weeks or at least 12 weeks.

Furthermore, the method may be repeatedly performed at least once per week or at least twice per week.

Furthermore, the method may be defined as a cycle, at least two cycles are repeatedly performed at least once per week or at least twice per week.

Furthermore, the cryotherapy apparatus may further comprise a temperature sensor detecting the temperature of the target area and a temperature regulator, wherein at operating the cryotherapy apparatus to spray the coolant, the temperature regulator may regulate a temperature of the coolant to reaching and maintaining the temperature of the target area to the target temperature based on the detected temperature of the target area and the target temperature.

According to the present disclosure, a cryotherapy apparatus of treating or preventing androgenetic alopecia of a human may comprise: a valve configured to allow a flow of a coolant which is provided from a coolant storage; a temperature regulator configured to regulate a temperature of the coolant; a nozzle configured to spray the coolant; a temperature sensor configured to measure a temperature of a target area which is a part of a human scalp; and a controller configured to the valve, the temperature sensor and the temperature regulator. wherein the controller is further configured to: set a target temperature to cool and maintain the target area to the set target temperature, receive, from the temperature sensor, the measured temperature of the target area, control the temperature regulator to regulate a temperature of the coolant based on the target temperature and the measured temperature of the target area, and control the valve to spray the coolant through the nozzle such that the temperature of the target area reaches to the target temperature and a cooling time of the target area is equal to or longer than a total cooling time period, in characterized that the target temperature may be equal to or less than 5 degrees Celsius and the total cooling time period may be equal to or longer than 17 seconds.

Furthermore, the controller, at the control the valve, may control the valve to spray the coolant continuously from a cooling starting time for the total cooling time period to a cooling terminating time for the total cooling time period, and may control the valve such that the target area is cooled to the target temperature and maintained its temperature for the total cooling time period.

Furthermore, the controller, at the control the valve, may control the valve to spray the coolant intermittently from a cooling starting time for the total cooling time period to a cooling terminating time for the total cooling time period, and may control the valve such that the target area is cooled to the target temperature and maintained its temperature for a first cooling time period, and then the target area is cooled again to the target temperature and maintained its temperature for a second cooling time period, wherein a sum of the first cooling time period and the second cooling time period may be same to the total cooling time period.

Furthermore, a cooling pause time period between the first time and the second time may be shorter than at least one of the first cooling time period and the second cooling time period.

Furthermore, the first cooling time period may be substantially identical with the second cooling time period.

Furthermore, the cooling pause time period may be predetermined such that cooling effect of the target area due to the first cooling time period does not disappear during the cooling pause time period.

Furthermore, each of the first cooling time period and the second cooling time period may be equal to or longer than 10 seconds.

Furthermore, the cooling pause time period may be equal to or shorter than 4 seconds.

Furthermore, the total cooling time period may be equal to or shorter than 40 seconds, 42 seconds, 44 seconds, 46 seconds, 48 seconds, or 50 seconds.

Furthermore, the total cooling time period may be equal to or shorter than 60 seconds, 63 seconds, 66 seconds, 69 seconds, 72 seconds, or 75 seconds.

Furthermore, the cooling the target area equal to or longer than 17 seconds which is the total cooling time at the 5 degrees Celsius which is the target temperature may activate at least one of FGF21, VEGF, CIRP, RBM3 and PGC 1a of the human.

Furthermore, the cooling the target area equal to or longer than 17 seconds which is the total cooling time at the 5 degrees Celsius which is the target temperature may induce an anagen phase of hair growth of the human scalp.

Furthermore, the coolant may be liquified carbon dioxide (CO2).

Furthermore, according to the present disclosure, a cryotherapy apparatus of treating or preventing alopecia of a human comprises a valve configured to allow a flow of a coolant which is provided from a coolant storage; a nozzle configured to spray the coolant; and a controller configured to the valve, wherein the controller is further configured to: set a target temperature to cool and maintain a target area, which is a part of a human scalp, to the set target temperature, and control the valve to spray the coolant through the nozzle such that a temperature of the target area reaches to the target temperature and a cooling time of the target area is equal to or longer than a total cooling time period, in characterized that the target temperature is equal to or less than 5 degrees Celsius and the total cooling time period is equal to or longer than 17 seconds.

Furthermore, the controller, at the control the valve, may control the valve to spray the coolant continuously from a cooling starting time for the total cooling time period to a cooling terminating time for the total cooling time period, and may control the valve such that the target area is cooled to the target temperature and maintained its temperature for the total cooling time period.

Furthermore, the controller, at the control the valve, may control the valve to spray the coolant intermittently from a cooling starting time for the total cooling time period to a cooling terminating time for the total cooling time period, and may control the valve such that the target area is cooled to the target temperature and maintained its temperature for a first cooling time period, and then the target area is cooled again to the target temperature and maintained its temperature for a second cooling time period, wherein a sum of the first cooling time period and the second cooling time period may be same to the total cooling time period.

Furthermore, a cooling pause time period between the first time and the second time may be shorter than at least one of the first cooling time period and the second cooling time period.

Furthermore, the first cooling time period may be substantially identical with the second cooling time period.

Furthermore, the cooling pause time period may be predetermined such that cooling effect of the target area due to the first cooling time period does not disappear during the cooling pause time period.

Furthermore, each of the first cooling time period and the second cooling time period may be equal to or longer than 10 seconds.

Furthermore, the cooling pause time period may be equal to or shorter than 4 seconds.

Furthermore, the total cooling time period may be equal to or shorter than 40 seconds, 42 seconds, 44 seconds, 46 seconds, 48 seconds, or 50 seconds.

Furthermore, the total cooling time period may be equal to or shorter than 60 seconds, 63 seconds, 66 seconds, 69 seconds, 72 seconds, or 75 seconds.

Furthermore, the cooling the target area equal to or longer than 17 seconds which is the total cooling time at the 5 degrees Celsius which is the target temperature may activate at least one of FGF21, VEGF, CIRP, RBM3 and PGC 1a of the human.

Furthermore, the cooling the target area equal to or longer than 17 seconds which is the total cooling time at the 5 degrees Celsius which is the target temperature may induce an anagen phase of hair growth of the human scalp.

Furthermore, the coolant may be liquified carbon dioxide (CO2).

### Difficulties in Alopecia Treatment through Scalp Cooling

Alopecia refers to a condition in which the number of hairs shed exceeds the number of hairs growing back, resulting in no more hair growing on body parts where hair should grow. A representative example of such alopecia includes scalp alopecia, where the number of hairs falling out of the hair follicles of the scalp exceeds the number of hairs growing from the hair follicles of the scalp.

Hair has a growth cycle that lasts from 2 to 6 years, and the hair growth cycle is composed of the anagen, catagen, and telogen phases. The anagen phase, the period during which hair grows, lasts from about 2 to 5 years. The catagen phase, the period during which hair stops growing and begins to fall out of the hair follicles, lasts from about 3 to 4 weeks. The telogen phase, the period during which hair completely falls out of the hair follicles and the hair follicles prepare to grow new hair, lasts from about 3 to 4 months.

Alopecia may occur due to abnormalities in the hair growth cycle. For example, when hair that has not fully grown falls out due to a shortened anagen phase, or when the hair life cycle ends before new hair begins to grow due to a shortened telogen phase (that is, no more hair grows), the number of hairs falling out of the scalp exceeds the number of hairs growing back. In such cases, alopecia may occur, resulting in overall hair loss from the scalp. In addition, due to abnormalities in the hair follicles or in the capillaries that supply nutrients to the hair follicles, nutrients supplied to the hair may be insufficient to grow hair length or thickness normally. In such cases, hair may not grow normally during the anagen phase, resulting in alopecia being occurred.

Therefore, alopecia is treatable by promoting hair growth so that hair can grow fully during the anagen phase or by extending the shortened anagen and telogen phases of hair so that the hair growth cycle can be repeatedly performed normally.

In the meantime, while scalp cooling is known to be effective for alopecia, it has only been known in the related art that scalp cooling may stimulate the arrector pili muscle of the hair follicles or relax the blood vessels around hair follicle cells to increase the blood flow rate, thereby inducing hair regrowth. In other words, the specific mechanisms involving the cooling conditions regarding how long and at what temperature the scalp should be cooled, and the like have not yet been identified. Therefore, treatment methods for meaningfully treating alopecia using cryotherapy remain undetermined.

Accordingly, all of the practitioners have used different methods of treating alopecia using cryotherapy, and the effects on patients subjected to alopecia treatment using cryotherapy also vary, leading to many cases where there is no effect. For this reason, it is challenging to secure patients' trust in the alopecia treatment effect using cryotherapy, which has been problematic. As a result, there has been a need to identify a specific mechanism to treat alopecia using cryotherapy.

In the meantime, the applicant of the present disclosure, who manufactures/sells a precise cooling control device configured to cool the skin (hereinafter referred to as a cryotherapy apparatus), has discovered a treatment method of treating alopecia by cooling the scalp using a cryotherapy apparatus.

Accordingly, in the present disclosure, an alopecia treatment method for treating alopecia using cryotherapy is to be described. In the meantime, alopecia, a disease described in the present disclosure, refers to scalp alopecia, where hair falls out of the scalp, as a representative form of alopecia but is not limited thereto. In other words, the treatment method described in the present disclosure is applicable not only to scalp alopecia but also to all types of alopecia with no more hair growing on body parts where hair should grow (for example, eyebrow alopecia).

In addition, those referred to as animals, to be described later, are living creatures biologically included in Animalia and include humans.

In the meantime, for the benefit of the following description in this specification, despite being expressed as “X degrees”, the temperature should be interpreted as “X degrees Celsius”. For example, in this specification, it should be interpreted as 5 degrees Celsius when described as 5 degrees and as 0 degrees Celsius when described as 0 degrees.

In addition, in the following description in this specification, being cooled to a target temperature of “X degrees (or X′ degrees or less, or X″ degrees or more and X′ degrees or less)” for a cooling time of “Y seconds (or Y′ seconds or more, or Y′ seconds or more and Y″ seconds or less)” refers to performing cooling on an area requiring alopecia treatment (hereinafter referred to as a target area), and performing cooling for a cooling time of Y seconds (or Y′ seconds or more, or Y′ seconds or more and Y″ seconds or less) so that the temperature of the target area is maintained at X degrees (or X′ degrees or less, or X″ degrees or more and X′ degrees or less) from the time the temperature of the target area reaches X degrees (or X′ degrees or less, or X″ degrees or more and X′ degrees or less). Thus, cooling the target area to a target temperature of “X degrees (or X′ degrees or less, or X″ degrees or more and X′ degrees or less)” for a cooling time of “Y seconds (or Y′ seconds or more, or Y′ seconds or more and Y″ seconds or less)” in this specification should be understood by the aforementioned description.

In the meantime, the term “target temperature” used in this specification refers to the target temperature of the target area, which is controlled to be maintained for the cooling time once the temperature of the target area is reached.

### Genetic Factors That Must Be Activated for Alopecia Treatment

It is known that the genetic factors of FGF21, VEGF, CIRP, RBM3, and PGC1α are involved in promoting hair growth or extending the hair growth cycle. Specifically, FGF21 helps regulate metabolism and anti-inflammation. Accordingly, scalp inflammation may be relieved, and oxidative damage to hair follicle cells due to active oxygen may occur less, thus promoting hair growth. VEGF, a genetic factor that promotes angiogenesis, can promote hair growth by increasing blood flow around hair follicles. CIRP is a genetic factor activated at low temperatures. CIRP may protect hair follicle cells from inflammation, heat, and oxidative damage and may maintain ribonucleic acid (RNA) stability to maintain the functions of hair follicle cells, thus promoting hair growth. RBM3 is also a genetic factor activated at low temperatures. RBM3 can promote protein synthesis and RNA stabilization to repair damaged hair follicles, and may extend the anagen phase of hair growth. PGC1a, a genetic factor that is also activated at low temperatures, can enhance the energy metabolism of hair follicle cells and prevent hair follicles from aging, thus maintaining the anagen phase of hair growth.

Thus, when at least one of the five genetic factors mentioned above is activated, depending on the activated genetic factor, hair growth can be promoted, or the anagen phase of hair growth may be extended or maintained, thus treating alopecia.

In the meantime, the applicant confirmed that the degree of activation of the five genetic factors mentioned above under the target temperature and cooling time, to be described later, is comparable to or greater than the degree of activation of the five genetic factors when minoxidil, a representative therapeutic in alopecia treatment, is applied on an alopecic area. Accordingly, the five genetic factors were confirmed to be activated sufficiently to the extent that alopecia is treatable. Furthermore, the applicant confirmed the induction of the anagen phase of hair growth in the area where alopecia occurred, under the target temperature and cooling time, to be described later.

Hereinafter, the cooling time and target temperature applied by the applicant to confirm the alopecia treatment effect through experiments are to be described. The above five genetic factors (FGF21, VEGF, CIRP, RBM3, and PGC1α) are referred to as alopecia treatment-related genetic factors hereinafter. Thus, unless otherwise mentioned, the term “alopecia treatment-related genetic factors” used in this specification may be understood as all five genetic factors or at least one of the five genetic factors. In addition, as used hereinafter, the term “effective in alopecia treatment” may refer to the induction of the anagen phase of hair growth or the degree of activation of at least one of the alopecia treatment-related genetic factors being comparable to or greater than that observed when applying minoxidil.

### Target Temperature and Cooling Time Proven Effective in Alopecia Treatment Through Animal Testing

The applicant cooled the dorsal side of a shaved mouse by spraying a coolant so that the temperature of the dorsal side of the mouse reached a specific target temperature and sprayed the coolant for a specific cooling time so that the temperature of the dorsal side of the mouse was maintained at the specific target temperature, thus conducting an experiment on the presence of the alopecia treatment effect on the dorsal side of the mouse.

As can be seen in Experimental Example 2, to be described later, when the target temperature was 5 degrees and the cooling time was 30 seconds, the applicant confirmed the induction of the anagen phase of hair growth on the dorsal side of the shaved mouse, leading to hair growth, and the activation of the alopecia treatment-related genetic factors.

In addition, as can be seen in Experimental Example 2, to be described later, when the target temperature was 5 degrees and the cooling time was 60 seconds, the applicant confirmed the induction of the anagen phase of hair growth on the dorsal side of the shaved mouse, leading to hair growth, and the activation of the alopecia treatment-related genetic factors.

In addition, as can be seen in Experimental Example 3 to be described later, when the target temperature was 0 degrees and the cooling time was 30 seconds, the applicant confirmed the induction of the anagen phase of hair growth on the dorsal side of the shaved mouse, leading to hair growth, and the activation of the alopecia treatment-related genetic factors.

In addition, as can be seen in Experimental Example 3, to be described later, when the target temperature was 0 degrees and the cooling time was 60 seconds, the applicant confirmed the induction of the anagen phase of hair growth on the dorsal side of the shaved mouse, leading to hair growth, and the activation of the alopecia treatment-related genetic factors.

Thus, according to the experimental results above, when cooling the animal skin to a target temperature of 5 degrees for a cooling time of 30 or 60 seconds, the alopecia treatment-related genetic factors were activated, confirming that alopecia was treatable. Furthermore, according to the experimental results above, when cooling the animal skin to a target temperature of 0 degrees for a cooling time of 30 or 60 seconds, the alopecia treatment-related genetic factors were activated, confirming that alopecia was treatable.

Moreover, taking into account the cooling time and target temperature leading to the activation of the alopecia treatment-related genetic factors and induction of the anagen phase of hair growth, as mentioned above, alopecia in an animal is treatable when cooling the animal skin by spraying the coolant such that the temperature of the animal skin to reaches the target temperature of 0 degrees or more and 5 degrees or less and the reached target temperature is maintained for 30 seconds or more and 60 seconds or less.

Experimental examples where the aforementioned target temperature and cooling time are specifically proven effective in alopecia treatment are to be described in detail later.

### Target Temperature and Cooling Time Capable of Treating Alopecia in Animal

According to the present disclosure, alopecia in an animal is treatable when cooling an area of the animal skin requiring alopecia treatment (hereinafter referred to as a target area) to 5 degrees or less for 30 seconds or more. This will be understood easily through Experimental Example 2 and the experimental results thereof, to be described later.

In addition, for example, alopecia in an animal is treatable, when cooling the target area to 4 degrees or less for 30 seconds or more. In addition, alopecia in an animal is treatable when cooling the target area to 3 degrees or less for 30 seconds or more. In addition, for example, alopecia in an animal is treatable when cooling the target area to 2 degrees or less for 30 seconds or more. In addition, alopecia in an animal is treatable when cooling the target area to 1 degree or less for 30 seconds or more. In addition, alopecia in an animal is treatable when cooling the target area to 0 degrees or less for 30 seconds or more. According to the aforementioned description, alopecia in an animal is treatable when cooling to a subzero temperature of less than 0 degrees for 30 seconds or more.

This is because cooling to 5 degrees or less for 30 seconds or more is confirmed to be effective in alopecia treatment in an animal through Experimental Examples 2 and the experimental results thereof. In addition, the lower the target temperature to which the animal skin is cooled, the greater the cooling energy reaching the hair follicles. Furthermore, the cooling effect on the hair follicles is more likely to be enhanced as the cooling energy increases. For this reason, the alopecia treatment effect of cooling is more likely to be enhanced. Therefore, alopecia in an animal is treatable even through cooling to a temperature of less than 5 degrees for 30 seconds or more.

In the meantime, alopecia in an animal is treatable when cooling the target area to 5 degrees or less for at least 30 seconds or more and 60 seconds or less. This will be understood easily in consideration of the cooling time that is proven effective for alopecia in Experimental Examples 2 and the experimental results thereof mentioned above.

In addition, for example, alopecia in an animal is treatable when cooling the target area to 4 degrees or less for at least 30 seconds or more and 60 seconds or less. In addition, alopecia in an animal is treatable when cooling the target area to 3 degrees or less for at least 30 seconds or more and 60 seconds or less. In addition, alopecia in an animal is treatable when cooling the target area to 2 degrees or less for at least 30 seconds or more and 60 seconds or less. In addition, alopecia in an animal is treatable when cooling the target area to 1 degree or less for at least 30 seconds or more and 60 seconds or less. In addition, alopecia in an animal is treatable when cooling the target area to 0 degrees or less for at least 30 seconds or more and 60 seconds or less. According to the aforementioned description, alopecia in an animal is treatable through cooling to a subzero temperature of less than 0 degrees for 30 seconds or more and 60 seconds or less. This is because, when lowering the target temperature of the target area, as mentioned above, the alopecia treatment effect is likely to be enhanced.

In the meantime, alopecia in an animal is treatable when cooling the target area to 0 degrees or more and 5 degrees or less for at least 30 seconds or more and 60 seconds or less. This will be understood easily in consideration of the target temperature that is proven effective for alopecia in Experimental Examples 2 and the experimental results thereof mentioned above.

In addition, for example, alopecia in an animal is treatable when cooling the target area to 0 degrees or more and 4 degrees or less for at least 30 seconds or more and 60 seconds or less. In addition, alopecia in an animal is treatable when cooling the target area to 0 degrees or more and 3 degrees or less for at least 30 seconds or more and 60 seconds or less. In addition, alopecia in an animal is treatable when cooling the target area to 0 degrees or more and 2 degrees or less for at least 30 seconds or more and 60 seconds or less. In addition, alopecia in an animal is treatable when cooling the target area to 0 degrees or more and 1 degree or less for at least 30 seconds or more and 60 seconds or less. This is because, when lowering the target temperature of the target area, as mentioned above, the alopecia treatment effect is likely to be enhanced.

In addition, 30 seconds or more may be interpreted as 28.5 seconds or more, or 29 seconds or more. This is because the results of experiments conducted for 30 seconds are predicted to be equally effective in alopecia treatment within a ±5% range under the doctrine of equivalents. In addition, 30 seconds or more may be interpreted as 27 seconds or more, 28 seconds or more, or 29 seconds or more. This is because the results of experiments conducted for 30 seconds are predicted to be equally effective in alopecia treatment within a ±10% range under the doctrine of equivalents. In addition, 30 seconds or more may be interpreted as 25.5 seconds or more, 26 seconds or more, 27 seconds or more, 28 seconds or more, or 29 seconds or more. This is because the results of experiments conducted for 30 seconds are predicted to be equally effective in alopecia treatment within a ±15% range under the doctrine of equivalents. In addition, 30 seconds or more may be interpreted as 24 seconds or more, 25 seconds or more, 26 seconds or more, 27 seconds or more, 28 seconds or more, or 29 seconds or more. This is because the results of experiments conducted for 30 seconds are predicted to be equally effective in alopecia treatment within a ±20% range under the doctrine of equivalents.

In addition, 60 seconds or less may be interpreted as 61 seconds or less, 62 seconds or less, or 63 seconds or less. This is because the results of experiments conducted for 60 seconds are predicted to be equally effective in alopecia treatment within a ±5% range under the doctrine of equivalents. In addition, 60 seconds or less may be interpreted as 61 seconds or less, 62 seconds or less, 63 seconds or less, 64 seconds or less, 65 seconds or less, or 66 seconds or less. This is because the results of experiments conducted for 60 seconds are predicted to be equally effective in alopecia treatment within a ±10% range under the doctrine of equivalents. In addition, 60 seconds or less may be interpreted as 61 seconds or less, 62 seconds or less, 63 seconds or less, 64 seconds or less, 65 seconds or less, 66 seconds or less, 67 seconds or less, 68 seconds or less, or 69 seconds or less. This is because the results of experiments conducted for 60 seconds are predicted to be equally effective in alopecia treatment within a ±15% range under the doctrine of equivalents. In addition, 60 seconds or less may be interpreted as 61 seconds or less, 62 seconds or less, 63 seconds or less, 64 seconds or less, 65 seconds or less, 66 seconds or less, 67 seconds or less, 68 seconds or less, 69 seconds or less, 70 seconds or less, 71 seconds or less, or 72 seconds or less. This is because the results of experiments conducted for 60 seconds are predicted to be equally effective in alopecia treatment within a ±20% range under the doctrine of equivalents.

### Spray-Type Cryotherapy Apparatus Used to Treat Alopecia in Animal

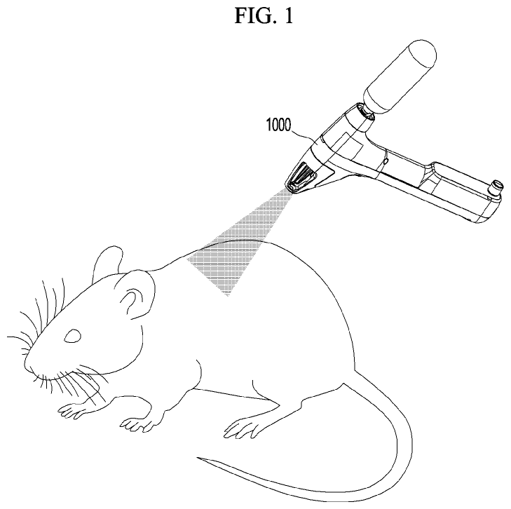

Referring to FIG. 1, an alopecia treatment method for an animal, described in this specification, may be performed by spraying a coolant on a target area using a spray-type cryotherapy apparatus 1000. For example, the spray-type cryotherapy apparatus 1000 may spray the coolant on the target area to cool the target area to a predetermined target temperature for a predetermined cooling time. In other words, by cooling the target area to the predetermined target temperature for the predetermined cooling time using the spray-type cryotherapy apparatus 1000, alopecia occurring in the target area is treatable.

Hereinafter, examples of the spray-type cryotherapy apparatus 1000 used to treat alopecia in an animal under the target temperature and cooling time, as mentioned above, are to be described. However, the alopecia treatment method, described in this specification, should not be limitedly interpreted as being performed only by the spray-type cryotherapy apparatus 1000, to be described later. The spray-type cryotherapy apparatus 1000, to be described later, is merely one example of the spray-type cryotherapy apparatus 1000 that can be used for alopecia treatment. In addition, it is apparent that the spray-type cryotherapy apparatus 1000 having the structure to be described below, as well as those having different structures, can be used for alopecia treatment described in the present disclosure as long as these apparatuses are capable of spraying the coolant on the target area requiring alopecia treatment, under the target temperature and cooling time for alopecia treatment.

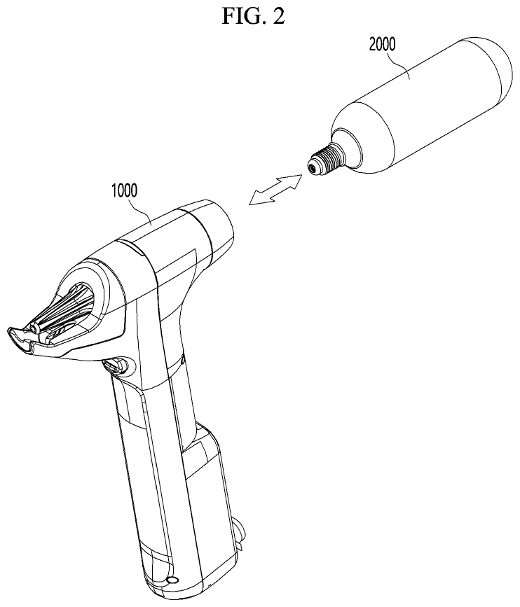

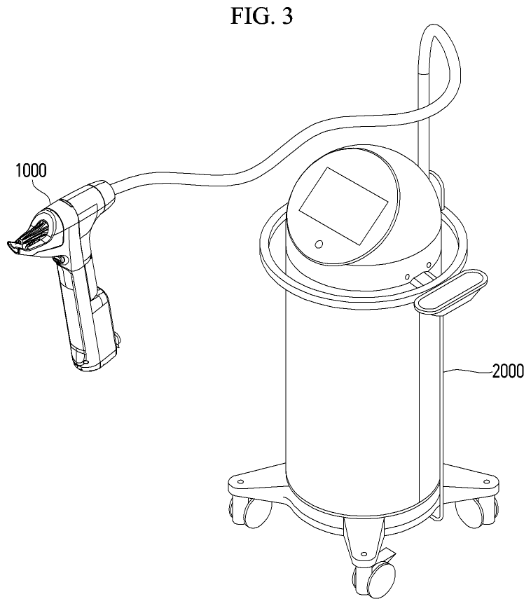

Referring to FIGS. 2 and 3, the spray-type cryotherapy apparatus 1000 may be designed to spray the coolant by receiving the coolant from a coolant storage 2000. Specifically, as illustrated in FIGS. 2 and 3, the coolant storage 2000 in which the coolant is stored is coupled to the spray-type cryotherapy apparatus 1000, and the coolant stored in the coolant storage 2000 may be sprayed to the outside through the coolant spraying device 1000.

For example, as can be seen in FIG. 2, the coolant storage 2000 may be a cartridge. A cartridge, a container designed to safely and efficiently store and transport the coolant, is highly portable. When such a cartridge is coupled to the coolant spraying device 1000, a coolant spraying system 100 may be portably used, thus increasing the convenience of use. The cartridge may store the coolant under a predetermined pressure. The pressure determined may be between about 35 bar and 1000 bar based on 0 °C to 40 °C and for example, may be between about 35 bar and 100 bar.

On the other hand, the coolant storage 2000 may be a tank. This tank may be a large-scale storage container configured to store a larger amount of the coolant. Referring to FIG. 3, the tank may be positioned within a housing configured to protect the tank. In addition, the tank within the housing may be fluidically connected to the coolant spraying device 1000 through a hose. In other words, the coolant stored in the tank is transferred to the coolant spraying device 1000 through the hose. The tank may limit the mobility of the coolant spraying device 1000 but may store a large amount of the coolant. Therefore, by reducing the frequency of refilling and/or replacing the coolant storage 2000, the operational continuity of the coolant spraying device 1000 may be improved.

### [1] First Example of Spray-Type Cryotherapy Apparatus 1000

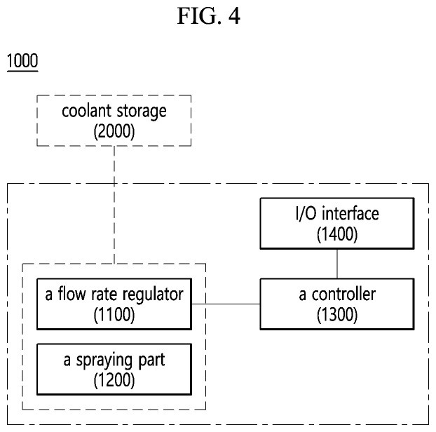

FIG. 4 is a view for describing one example of the spray-type cryotherapy apparatus 1000 used in the alopecia treatment method described in the present disclosure.

According to one example, the spray-type cryotherapy apparatus 1000 may include a flow rate regulator 1100, a spraying part 1200, a controller 1300, and an inlet/outlet (I/O) interface 1400. In this case, the flow rate regulator 1100 and the spraying part 1200 are fluidically connected to the coolant storage 2000 through at least one conduit. For example, the flow rate regulator 1100 and the spraying part 1200 may be fluidically connected to each other through a first conduit, and the flow rate regulator 1100 and the coolant storage 2000 may be fluidically connected to each other through a second conduit. For example, when the coolant storage 2000 is a cartridge, as the cartridge is coupled to a coolant storage connector (not shown) of the spray-type cryotherapy apparatus 1000, the cartridge may be fluidically connected to the flow rate regulator 1100 through the second conduit.

For example, when the coolant storage 2000 is a tank, as a third conduit fluidically connected to the tank is coupled to the coolant storage connector (not shown) of the spray-type cryotherapy apparatus 1000, the tank and the flow rate regulator 1100 may be fluidically connected through the second and third conduits. In this case, the second conduit may be a conduit fluidically connecting the flow rate regulator 1100 and the coolant storage connector.

The flow rate regulator 1100, a device configured to control the flow of the coolant, may allow the coolant to be transferred to the spraying part 1200. For example, the flow rate regulator 1100 may be a valve. When the valve is opened, the coolant may be transferred toward the spraying part 1200 from the valve. When the valve is closed, the coolant that has not yet passed through the valve fails to be transferred toward the spraying part 1200. The amount of cooling energy provided per unit time to the target area may be adjusted using the flow rate regulator 1100. For example, when the opening/closing cycle of the valve is adjusted, the amount of coolant sprayed per unit time on the target area may be adjusted. In the meantime, the valve may be a solenoid valve, but is not limited thereto. In other words, any valve capable of regulating the flow rate of the coolant can be used as the flow rate regulator 1100 according to the present disclosure. In the present disclosure, the flow rate regulator 1100 may be referred to as a valve.

The spraying part 1200 may include a structure configured to spray the coolant. Specifically, the spraying part 1200 may form a flow path through which the coolant flows by extending from one end to the other, but the flow path may include a relatively narrow part. The coolant passing through the spraying part 1200 may have increased pressure while passing through the narrow part, may have a rapidly decreased temperature due to expansion caused by the pressure drop when sprayed into the atmospheric pressure, and may be sprayed at a high speed. In the present disclosure, the spraying part 1200 may be referred to as a nozzle. However, the technical idea of the present disclosure is not limited thereto, and the spraying part 1200 may be understood as a configuration including a flow path to spray the coolant to the outside of the coolant spraying device 1000. In the meantime, the spraying part 1200 is attachable to and detachable from the coolant spraying device 1000. For example, the spraying part 1200 may be coupled to the coolant spraying device 1000 through a spraying part-coupling part (not shown) or may be detached from the coolant spraying device 1000. Alternatively, the spraying part 1200 may be physically connected to the coolant spraying device 1000 and configured as one body.

The I/O interface 1400 may include an input part and an output part. The input part provides an interface, such as a switch, to receive user input and, therefore, may be referred to as an input interface. The input part may receive user input. For example, a user may input a target temperature and cooling time for alopecia treatment into the controller 1300 through the input part. In this case, a user may also input one target temperature and one cooling time, but may input a plurality of target temperatures and a plurality of cooling times. The method of treating alopecia using the spray-type cryotherapy apparatus 1000 when a user inputs a plurality of target temperatures and a plurality of cooling times is to be described later.

In the meantime, the target temperature input through the I/O interface 1400 is the input target temperature that the target area is intended to reach and maintain and, therefore, may also be referred to as the target temperature.

The output part provides a user with an interface to output a variety of information for the use of the coolant spraying device 1000 and, therefore, may be referred to as an output interface. For example, the output part may include a display, and a variety of information for setting the target temperature, the cooling time, and the like may be output through the display. In addition, during the operation of the coolant spraying device 1000, information such as the real-time temperature of the target area or the total time of the coolant being sprayed may be output.

The controller 1300 may be electrically connected to the flow rate regulator 1100 and the I/O interface 1400. In addition, the controller 1300 may control the configurations electrically connected to the controller 1300. For example, the controller 1300 may set a target temperature and/or a cooling time on the basis of the input signal received from the I/O interface 1400. In addition, when a coolant spraying command signal is received from the I/O interface 1400, the controller 1300 may control the opening/closing of the flow rate regulator 1100 on the basis of the set target temperature and/or cooling time, thus spraying the coolant. For example, the controller 1300 may count the cooling time when the target area reaches the target temperature. In addition, the controller 1300 may provide an alert to a user through the I/O interface 1400 upon expiration of the cooling time count or may control the opening/closing of the flow rate regulator 1100 to stop the coolant spraying.

For example, the controller 1300 may store at least one of the opening/closing cycle, the opening/closing time, and the number of openings/closings per unit time, per the target temperature and/or the cooling time, and may retrieve at least one of the opening/closing cycle, opening/closing time, and number of openings/closings per unit time of the flow rate regulator 1100, on the basis of the set target temperature and/or cooling time. In addition, the controller 1300 may control the opening/closing of the flow rate regulator 1100 according to at least one of the retrieved opening/closing cycle, opening/closing time, and number of openings/closings per unit time to spray the coolant.

In the meantime, the spray-type cryotherapy apparatus 1000, according to the present disclosure, may further include a temperature sensor (not shown). The temperature sensor (not shown) may obtain temperature information of the target area by measuring the temperature of the target area where the coolant is sprayed. In addition, the temperature sensor (not shown) may provide the obtained temperature information to the controller 1300. In the meantime, one or more temperature sensors (not shown) may be included in the spray-type cryotherapy apparatus 1000. For example, one temperature sensor may measure the temperature of the target area to provide the obtained temperature information to the controller 1300, or each of the two or more temperature sensors may measure the temperature of the target area to provide the obtained temperature information to the controller 1300. When the spray-type cryotherapy apparatus 1000, according to the present disclosure, includes the temperature sensor (not shown), a method of controlling the opening/closing of the flow rate regulator 1100 by the controller 1300, on the basis of the set target temperature and/or cooling time, may vary. For example, while the coolant is sprayed, the controller 1300 may obtain temperature information by measuring the temperature of the target area where the coolant is sprayed through the temperature sensor (not shown) and may control the opening/closing of the flow rate regulator 1100 on the basis of the obtained temperature information and the set target temperature. In other words, the controller 1300 may determine at least one of the opening/closing cycle, opening/closing time, and number of openings/closings per unit time of the flow rate regulator 1100, on the basis of the obtained temperature information and the set target temperature, to control the opening/closing of the flow rate regulator 1100, thus spraying the coolant.

In the meantime, through the opening/closing of the aforementioned flow rate regulator 1100, the controller 1300 may control the coolant spraying so that the temperature of the target area reaches the target temperature. When the temperature of the target area is determined to reach the set target temperature, the controller 1300 may control the flow rate regulator 1100 so that the temperature of the target area is maintained at the set target temperature for the set cooling time, thus adjusting the temperature of the target area. In addition, when the set cooling time has elapsed, an alert may be provided to a user, or the opening/closing of the flow rate regulator 1100 may be controlled to stop the coolant spraying.

In the meantime, the spray-type cryotherapy apparatus 1000, according to FIG. 4, may further include a memory (not shown). The memory (not shown) may be where basic programs for operating the spray-type cryotherapy apparatus 1000 in response to user commands are stored. In addition, the memory (not shown) may store combinations of the target temperature and the cooling time for treating alopecia in an animal. A user may select any one of the combinations stored in the memory (not shown) through the input part to input the target temperature and the cooling time into the controller 1300. Alternatively, the memory (not shown) may store treatment information of a patient with alopecia in addition to the combinations above. When a user selects a patient with alopecia through the input part, the controller 1300 may load an appropriate combination of target temperature and cooling time for the patient with alopecia according to the corresponding treatment information from the memory (not shown). In the meantime, the treatment information is not stored in the memory (not shown), and when a user inputs the treatment information through the input part, the controller 1300 may select an appropriate combination of target temperature and cooling time for the input treatment information from the memory (not shown).

However, a user may also input the target temperature and the cooling time that differ from those of the combinations stored above into the controller 1300 through the input part. In addition, the memory (not shown) may not store combinations of target temperature and cooling time. In such cases, a user may control the input part to input the target temperature and the cooling time into the controller 1300. In the meantime, the memory (not shown) may store only one target temperature and cooling time. In such cases, when a user transmits a coolant spraying command signal to the controller 1300 through the input part without selecting or inputting a separate target temperature and cooling time through the input part, the spray-type cryotherapy apparatus 1000 may spray the coolant on the target area according to one target temperature and one cooling time stored above.

### [2] Second Example of Spray-type Cryotherapy Apparatus 1000

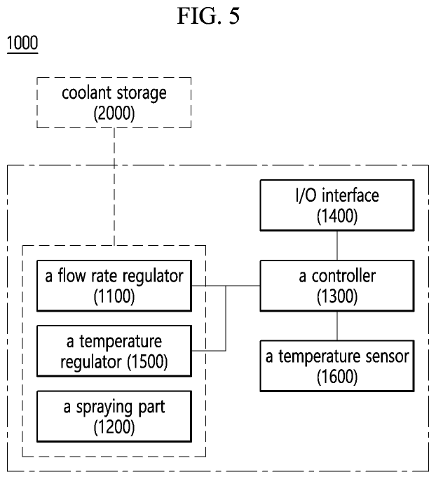

FIG. 5 is a view for describing another example of the spray-type cryotherapy apparatus 1000 used in the alopecia treatment method described in the present disclosure.

According to another example, the spray-type cryotherapy apparatus 1000 may include a flow rate regulator 1100, a temperature regulator 1500, a spraying part 1200, a controller 1300, a temperature sensor 1600, and an I/O interface 1400. In this case, the flow rate regulator 1100, the temperature regulator 1500, and the spraying part 1200 are fluidically connected to the coolant storage 2000 through at least one conduit. For example, the flow rate regulator 1100 and the temperature regulator 1500 may be fluidically connected through a first conduit, while the temperature regulator 1500 and the spraying part 1200 may be fluidically connected through a second conduit. For another example, the flow rate regulator 1100 and the temperature regulator 1500 may be fluidically connected through the first conduit, while the flow rate regulator 1100 and the spraying part 1200 may be fluidically connected through the second conduit. In addition, the coolant storage 2000 may be fluidically connected to the flow rate regulator 1100 or the temperature regulator 1500 through a third conduit. For example, when the coolant storage 2000 is a cartridge, as the cartridge is coupled to a coolant storage connector (not shown) of the spray-type cryotherapy apparatus 1000, the cartridge may be fluidically connected to the flow rate regulator 1100 or the temperature regulator 1500 through the third conduit. For another example, when the coolant storage 2000 is a tank, as a fourth conduit fluidically connected to the tank is coupled to the coolant storage connector (not shown) of the spray-type cryotherapy apparatus 1000, the tank and the flow rate regulator 1100 may be fluidically connected through the third and fourth conduits.

In this case, the third conduit may be a conduit fluidically connecting the flow rate regulator 1100 or the temperature regulator 1500 with the coolant storage connector.

In the meantime, the flow rate regulator 1100, the spraying part 1200, the temperature sensor 1600, and the I/O interface 1400, among the configurations included in the spray-type cryotherapy apparatus 1000, are the same as the flow rate regulator 1100, the spraying part 1200, the temperature sensor (not shown), and the I/O interface 1400 in the aforementioned “First Example of Spray-Type Cryotherapy Apparatus 1000”, respectively. Therefore, redundant descriptions thereof are to be omitted.

The temperature regulator 1500 is a device configured to adjust the temperature of the coolant passing through the temperature regulator 1500. For example, the temperature regulator 1500 may heat the coolant under the control of the controller 1300 to adjust the temperature of the coolant. In such cases, the temperature regulator 1500 may be referred to as a heater. For another example, the temperature regulator 1500 may expand or compress the coolant under the control of the controller 1300, thereby adjusting the temperature of the coolant. In such cases, the temperature regulator 1500 may be referred to as a pressure regulator.

The controller 1300 may be electrically connected to the flow rate regulator 1100, the temperature regulator 1500, the temperature sensor 1600, and the I/O interface 1400. In addition, the controller 1300 may control the configurations electrically connected to the controller 1300. For example, the controller 1300 may set a target temperature and/or a cooling time on the basis of the input signal received from the I/O interface 1400. In addition, when a spraying command signal is received from the I/O interface 1400, the controller 1300 may control the opening/closing of the flow rate regulator 1100 such that the coolant is sprayed.

In addition, the controller 1300 may control the temperature regulator 1500 to adjust the temperature of the coolant. For example, the controller 1300 may supply power to the temperature regulator 1500 so that thermal energy for heating the coolant can be generated in the temperature regulator 1500. Specifically, when the power supplied to the temperature regulator 1500 increases, the thermal energy generated in the temperature regulator 1500 may increase. For another example, the controller 1300 may control the temperature regulator 1500 such that the coolant is expanded or compressed in the temperature regulator 1500, thus adjusting the temperature of the coolant. For example, the controller 1300 may control the temperature regulator 1500 such that the Joule-Thomson effect allows the temperature of the coolant to decrease by expanding the coolant or allows the temperature of the coolant to increase by compressing the coolant.

In the meantime, the controller 1300 may control the temperature regulator 1500 on the basis of the temperature information received from the temperature sensor 1600 and the set target temperature. For a further detailed description in this regard, the controller 1300 may set the target temperature and/or the cooling time. For example, when the spraying command signal is received from the I/O interface 1400, the controller 1300 may control the opening/closing of the flow rate regulator 1100 to spray the coolant. In the meantime, while the coolant is sprayed, the controller 1300 may obtain the temperature information by measuring the temperature of the target area where the coolant is sprayed through the temperature sensor 1600 and may adjust power such that the power determined on the basis of the obtained temperature information and the set target temperature is applied to the temperature regulator 1500. Alternatively, the temperature regulator 1500 may be controlled such that the coolant is expanded or compressed on the basis of the obtained temperature information and the set temperature.

In the meantime, unlike shown in FIG. 5, the spray-type cryotherapy apparatus 1000 may not include the temperature sensor 1600. In such cases, the controller 1300 may store the amount of heat or power required to be applied according to the target temperature and/or the cooling time or may store the pressure of the coolant required to be adjusted according to the target temperature and/or the cooling time. In addition, the controller 1300may adjust, on the basis of the set target temperature and cooling time, the amount of heat or power required for the temperature regulator 1500 to heat the coolant or may control the coolant to expand or compress.

In the meantime, through the control of the aforementioned temperature regulator 1500, the controller 1300 may adjust the temperature of the coolant so that the temperature of the target area reaches the set target temperature, and may control the opening/closing of the flow rate regulator 1100 so that the coolant is sprayed on the target area. When the temperature of the target area is determined to reach the set target temperature, the controller 1300 may adjust the temperature of the coolant through the temperature regulator 1500 so that the temperature of the target area is maintained at the set target temperature for the set cooling time while controlling the opening/closing of the flow rate regulator 1100 so that the coolant is sprayed on the target area. In addition, when the cooling time has elapsed, the controller 1300 may provide an alert to a user or may control the opening/closing of the flow rate regulator 1100 to stop the coolant spraying.

In the meantime, the spray-type cryotherapy apparatus 1000, according to FIG. 5, may also further include a memory (not shown). The descriptions of the memory (not shown) and the operation of the memory (not shown) are the same as those described in [First Example of Spray-Type Cryotherapy Apparatus 1000] and, therefore, are to be omitted.

### [3] Coolant Used in Spray-type Cryotherapy Apparatus

The coolant may refer to a substance capable of applying cooling energy to a target area. For example, carbon dioxide (CO2), liquid nitrogen (LN), oxygen (O2), nitrous oxide (N2O), nitrogen monoxide (NO), hydrofluorocarbon (HFC)-based materials, methane (CH4), perfluorinated compounds (PFCs), SF6, krypton, helium-3, ethyl chloride, dimethyl ether, chlorofluoromethane, chloromethane, propane, butane, cooling water, cooling gas, air, or a combination thereof can be used as the coolant.

In this case, carbon dioxide is preferably selected as the coolant for alopecia treatment. When carbon dioxide reaches the scalp, blood circulation in the scalp is promoted, thus increasing the blood flow rate around the hair follicles increases and the amount of oxygen delivered to the scalp. Accordingly, as nutrients and oxygen are supplied to hair follicle cells, the metabolism thereof becomes active, thus promoting hair growth. In addition, carbon dioxide relieves inflammation in the scalp and removes impurities and sebum from the scalp, helping create a scalp environment that allows hair to grow more healthily. Thus, carbon dioxide is preferably used as the coolant of the spray-type cryotherapy apparatus 1000 for alopecia treatment.

### Alopecia Treatment Method for Animal

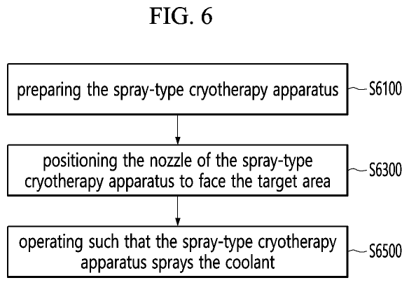

With reference to FIGS. 6 to 8, the method of treating alopecia in an animal using the aforementioned spray-type cryotherapy apparatus 1000 or spray-type cryotherapy apparatuses having different structures is to be described. FIGS. 6 to 8 are views for describing an alopecia treatment act of a user of the spray-type cryotherapy apparatus 1000, configured to treat alopecia in an animal, using the spray-type cryotherapy apparatus 1000.

Referring to FIG. 6, a user may prepare the aforementioned spray-type cryotherapy apparatus 1000 (S6100). Then, the user may position the nozzle of the spray-type cryotherapy apparatus 1000 to face the target area (S6300). In this case, the target area, a part of the animal skin, may be where alopecia occurs. The user may operate the spray-type cryotherapy apparatus 1000 so that the coolant is sprayed on the target area while the nozzle faces the target area (S6500).

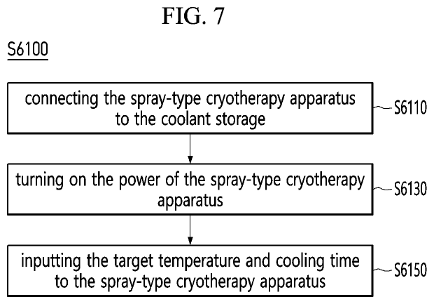

FIG. 7 is a view for describing one example of S6100 in which the user prepares the spray-type cryotherapy apparatus 1000. Referring to FIG. 7, the user may connect the spray-type cryotherapy apparatus 1000 to the coolant storage 2000 (S6110). Embodiments in which the spray-type cryotherapy apparatus 1000 and the coolant storage 2000 are connected have already been mentioned above by distinguishing the cases where the coolant storage 2000 is a cartridge or a tank. Therefore, a detailed description thereof is to be omitted.

The user may turn on the power of the spray-type cryotherapy apparatus 1000 (S6130). When turning on the power of the spray-type cryotherapy apparatus 1000, the I/O interface 1400 and/or the controller 1300 may be set so that a target temperature and a cooling time can be input. In addition, the user may input the target temperature and cooling time into the controller 1300 through the I/O interface 1400 to complete the preparation of the spray-type cryotherapy apparatus 1000 (S6150). In this case, the input target temperature and cooling time may be any one of the combinations of target temperature and cooling time described in [Target Temperature and Cooling Time Capable of Treating Alopecia in Animal]. In addition, the combinations of target temperature and cooling time may be stored in the memory (not shown), and the user may select any one of the stored combinations through the I/O interface 1400. Alternatively, the user may select a patient with alopecia or treatment information of the patient with alopecia, rather than inputting the target temperature and the cooling time in S6150. When the user selects the patient with alopecia or the treatment information of the patient with alopecia, the spray-type cryotherapy apparatus 1000 may select an appropriate combination of target temperature and cooling time, as mentioned above. In the meantime, when the target temperature and the cooling time are set in one combination, as mentioned above, S6150 may be omitted.

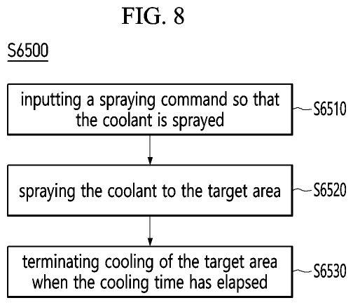

FIG. 8 is a view for describing one example of S6500 in which the user operates the spray-type cryotherapy apparatus 1000. Referring to FIG. 8, the user may set the I/O interface 1400 or press the input button of the spray-type cryotherapy apparatus 1000 to input a spraying command so that the coolant is sprayed from the spray-type cryotherapy apparatus 1000 (S6510). In this case, when the spraying command is input, the coolant is continuously sprayed on the target area through the spray-type cryotherapy apparatus 1000 (S6520). For example, the spray-type cryotherapy apparatus 1000 continuously sprays the coolant on the target area so that the target area reaches the target temperature. Once the temperature of the target area reaches the target temperature, the spray-type cryotherapy apparatus 1000 continuously sprays the coolant on the target area so that the temperature of the target area is maintained at the target temperature for the cooling time thereafter. In this case, the target temperature and cooling time may be any one of the combinations of target temperature and cooling time described in [Target Temperature and Cooling Time Capable of Treating Alopecia in Animal].

When the spray-type cryotherapy apparatus 1000 sounds an alarm after the cooling time has elapsed, the user may terminate cooling of the target area by allowing the nozzle of the spray-type cryotherapy apparatus 1000 to face an area (for example, air or other skin areas) other than the target area. Alternatively, when the spray-type cryotherapy apparatus 1000 sounds an alarm after the cooling time has elapsed, the user may terminate cooling of the target area by turning the power of the spray-type cryotherapy apparatus 1000 off or stopping the operation of the spray-type cryotherapy apparatus 1000. Alternatively, when the cooling time has elapsed, the spray-type cryotherapy apparatus 1000 may terminate cooling of the target area by stopping the coolant spraying.

### Target Temperature and Cooling Time Capable of Treating Alopecia in Human

The combinations of target temperature and cooling time for treating alopecia in an animal have been described above in light of Experimental Example 2 and the experimental results thereof. In the meantime, each human is also one animal species, meaning that when spraying the coolant on an alopecic area in a human, alopecia is treatable according to the target temperature and the cooling time, as mentioned above. However, the human scalp exhibits different properties from other skin areas of a human or the skin of other animals. Considering such properties of the human scalp, alopecia in a human is treatable when cooling the human scalp to 0 degrees or less for 18 seconds or more. This is because the target temperature and the cooling time are set such that the same amount of cooling energy transferred to mouse hair follicles when cooling the mouse skin surface to 5 degrees or less for a cooling time of 30 seconds or more is transferred to human hair follicles.

In the meantime, the conclusion that alopecia in a human is treatable when cooling the human scalp to 0 degrees or less for 18 seconds or more can be seen through Experimental Example 4, to be described later.

Experimental Examples 2 and the experimental results thereof has demonstrated that spraying the coolant on the dorsal side of mice is effective in alopecia treatment. In the meantime, mouse hair follicles and human hair follicles have quite similar tissue structures, such as the hair bulb and hair papilla. Therefore, when the same amount of cooling energy transferred to mouse hair follicles under the target temperature and cooling time, confirmed to be effective in alopecia treatment, is transferred to human hair follicles, the cooling effects observed on the hair follicles are likely to be the same. In addition, the cooling effect being equally observed in human and mouse hair follicles means being equally effective in alopecia treatment in a human when the same amount of cooling energy reaches human hair follicles.

To calculate the target temperature at which and the cooling time for which the cooling energy transferred to mouse hair follicles is transferred to human hair follicles, the amount of heat transferred per unit area and unit time from a first point to a second point may be obtained using Fourier's law of heat conduction. Fourier's law of heat conduction is as follows.

q=−k*(dT/dL) [Equation 1]

In this case, q refers to the amount of heat transferred per unit area and unit time from the first point to the second point, k refers to the thermal conductivity, dT refers to the temperature difference between the first and second points, and dL refers to the distance between the first and second points.

Using this equation, the cooling energy reaching mouse hair follicles when cooling the temperature of the mouse skin surface to 5 degrees for 30 seconds is calculated. When calculating the cooling energy reaching the mouse hair follicles, the first point refers to the mouse skin surface, while the second point refers to the mouse hair follicles. Thus, dT refers to the temperature difference between the mouse skin surface and the mouse hair follicles. In addition, dL refers to the distance between the mouse skin surface and the mouse hair follicles. Furthermore, k represents the thermal conductivity of the mouse skin.

Hereinafter, to know dL, dT, and k values, the properties of the mouse skin are to be examined, and representative values for dL, dT, and k are to be determined therethrough. When no cooling is performed, the initial temperature of the hair follicles may be similar to the body temperature of a mouse. Accordingly, the initial temperature of the mouse hair follicles before performing cooling may be between 36 degrees and 39 degrees, which is typically between 36.5 degrees and 37.5 degrees. Thus, 37 degrees, which is the median value of 36.5 degrees and 37.5 degrees, is determined as the representative value for the initial temperature of the mouse hair follicles.

In the meantime, the distance from the mouse skin surface to the hair follicles is known to be between 1 mm and 2.5 mm, and hair follicles are known to be distributed primarily between 1 mm and 2 mm from the skin surface. Thus, 1.5 mm, which is the median value of 1 mm and 2 mm, is determined as the representative value for the distance from the mouse skin surface to the hair follicles.

In addition, the thermal conductivity of the mouse skin is known to be between 0.1 W/mK and 0.4 W/mK, which is typically known to be between 0.18 W/mK and 0.22 W/mK. Thus, 0.2 W/mK, which is the median value of 0.18 W/mK and 0.22 W/mK, is determined as the representative value for the thermal conductivity of the mouse skin.

In summary, the thermal conductivity of the mouse skin is 0.2 W/mK, the distance from the mouse skin surface to the hair follicles is 1.5 mm, and the initial temperature of the mouse hair follicles is 37 degrees.

When using the representative values determined above as inputs to [Equation 1], q=−(0.2)*(37−5)/(1.5*10\hat{ }−3)=−4300 (W/m²) is derived. In other words, the amount of cooling energy transferred per unit area and per unit time from the mouse skin to the mouse hair follicles is 4300 (W/m²). The total amount of cooling energy reaching the mouse hair follicles per unit area for 30 seconds is −4300*30=−129,000 (W/m²).

Using this, the time required for a cooling energy of −129,000 (W/m²) to be transferred to human hair follicles when cooling the surface temperature of the human scalp to a target temperature of 0 degrees or less is to be calculated. To this end, in [Equation 1], the first point is defined to refer to the human scalp surface, while the second point is defined to refer to the human hair follicles. Thus, dT refers to the temperature difference between the human scalp surface and the human hair follicles, and dL refers to the distance between the human scalp surface and the human hair follicles. In addition, k represents the thermal conductivity of the human scalp.

Furthermore, to know dL, dT, and k values, the properties of the human skin are examined, and representative values for dL, dT, and k are determined therethrough. When no cooling is performed, the initial temperature of the human hair follicles may be similar to the body temperature of a human. Accordingly, the initial temperature of the human hair follicles before performing cooling may be between 35.5 degrees and 38 degrees, which is typically known to be between 36.5 degrees and 37.5 degrees. Thus, 37 degrees, which is the median value of 36.5 degrees and 37.5 degrees, is determined as the representative value for the initial temperature of the human hair follicles.

In the meantime, the distance from the human scalp surface to the hair follicles is known to be between 1 mm and 3 mm, and hair follicles are known to be distributed primarily between 1.5 mm and 2.5 mm from the scalp surface. Thus, 2 mm, which is the median value of 1.5 mm and 2.5 mm, is determined as the representative value for the distance from the human scalp surface to the hair follicles.

In addition, the thermal conductivity of the human scalp is known to be between 0.2 W/mK and 0.6 W/mK, which is typically known to be between 0.35 W/mK and 0.45 W/mK. Thus, 0.4 W/mK, which is the median value of 0.35 W/mK and 0.45 W/mK, is determined as the representative value for the thermal conductivity of the human scalp.

When using the representative values determined above as inputs to [Equation 1], q=−(0.4)*(37−0)/(2*10\hat{ }−3)=−7400 (W/m²) is derived. In other words, the amount of cooling energy transferred per unit area and unit time from the human scalp to the human hair follicles is −7400 (W/m²). Thus, in order for a cooling energy of −129,000 (W/m²) to be transferred to the human scalp, it is seen that a cooling time of −129,000/−7,400=17.43 seconds is required, and conservatively, a cooling time of 18 seconds is required.

Accordingly, those skilled in the art can easily predict that cooling the scalp area for treating alopecia in a human to a target temperature of 0 degrees or less for a cooling time of 18 seconds or more is likely to be effective in alopecia treatment in a human. Thus, the combination of target temperature and cooling time for treating alopecia in a human may be a temperature of 0 degrees or less and a cooling time of 18 seconds or more.

In this case, the term “scalp area for treating alopecia in a human” may be referred to as the term “target area” in the description to be disclosed later. In other words, the term “target area” may be a part of the human scalp and may refer to the scalp area for treating alopecia. It should be noted that the term “target area” is distinguished from those referring to parts of the skin area for treating alopecia in an animal in the aforementioned description of alopecia treatment in an animal. In other words, the definition of the term “target area” should be interpreted differently depending on the subject of alopecia treatment.

In the meantime, cooling the target area of a human to a target temperature of 5 degrees or less for a cooling time of 21 seconds or more is effective in alopecia treatment in a human. In addition, cooling the target area of a human to a target temperature of 4 degrees or less for a cooling time of 20 seconds or more is effective in alopecia treatment in a human. In addition, cooling the target area of a human to a target temperature of 3 degrees or less for a cooling time of 19 seconds or more is effective in alopecia treatment in a human. In addition, cooling the target area of a human to a target temperature of 2 degrees or less for a cooling time of 19 seconds or more is effective in alopecia treatment in a human. In addition, cooling the target area of a human to a target temperature of 1 degree or less for a cooling time of 18 seconds or more is effective in alopecia treatment in a human. In addition, cooling the target area of a human to a target temperature of −1 degrees or less for a cooling time of 17 seconds or more is effective in alopecia treatment in a human. The aforementioned combinations of target temperature and cooling time are configured to allow a cooling energy of −129,000 (W/m²) to reach human hair follicles in the same manner as described above.

In the meantime, alopecia in a human is treatable when cooling the target area of a human to a target temperature of 0 degrees or less for a cooling time of 18 seconds or more and 35 seconds or less. In this regard, Experimental Example 2 has demonstrated that cooling the temperature of the mouse skin surface to 5 degrees for 60 seconds is also effective in alopecia treatment, which can be seen when calculating the cooling energy transferred by cooling the mouse skin to a target temperature of 5 degrees for 60 seconds. The cooling energy transferred per unit area and unit time to hair follicles when cooling the mouse skin surface to 5 degrees is −6400 (W/m²), so the cooling energy transferred to the hair follicles for 60 seconds per unit area is −258,000 (W/m²). In the meantime, the cooling energy transferred per unit area and unit time to hair follicles when cooling the human scalp surface to 0 degrees is −7400 (W/m²). Therefore, in order for a cooling energy of −258,000 (W/m²) to be transferred to the human scalp, it is seen that a cooling time of 34.86 seconds is required, and conservatively, a cooling time of 35 seconds is required.

In addition, cooling the target area of a human to a target temperature of 5 degrees or less for a cooling time of 21 seconds or more and 42 seconds or less is effective in alopecia treatment in a human. In addition, cooling the target area of a human to a target temperature of 4 degrees or less for a cooling time of 20 seconds or more and 40 seconds or less is effective in alopecia treatment in a human. In addition, according to the aforementioned method, cooling the target area of a human to a target temperature of 3 degrees or less for a cooling time of 19 seconds or more and 38 seconds or less is effective in alopecia treatment in a human. In addition, according to the aforementioned method, cooling the target area of a human to a target temperature of 2 degrees or less for a cooling time of 19 seconds or more and 38 seconds or less is effective in alopecia treatment in a human. In addition, cooling the target area of a human to a target temperature of 1 degree or less for a cooling time of 18 seconds or more and 36 seconds or less is effective in alopecia treatment in a human. In addition, cooling the target area of a human to a target temperature of-1 degrees or less for a cooling time of 17 seconds or more and 34 seconds or less is effective in alopecia treatment in a human. The aforementioned combinations of target temperature and cooling time are configured to allow a cooling energy of −258,000 (W/m²) to reach human hair follicles in the same manner as described above.

In the meantime, cooling the human scalp skin to a target temperature of 0 degrees or less for a cooling time of 20 seconds or more is effective in alopecia treatment in a human. This can be seen through the calculation according to [Equation 1], as mentioned above, on the basis of the conclusion that Experimental Example 3 has demonstrated that cooling the mouse skin surface to 0 degrees or less for a cooling time of 30 seconds or more is effective in alopecia treatment. The cooling energy transferred to mouse hair follicles when cooling the mouse skin surface to 0 degrees or less for a cooling time of 30 seconds or more according to [Equation 1] is calculated as follows: q=−0.2*(37−0)/(1.5*10\hat{ }−3)=−4933 (W/m²). Thus, the cooling energy transferred for 30 seconds is −4933*30=−148,000 (W/m²).

The cooling energy transferred per unit area and unit time when cooling the human scalp surface to 0 degrees has already been calculated above as −7400 (W/m²). Therefore, the cooling time required to be effective for alopecia when cooling the human scalp surface to 0 degrees is −148,000/−7,400=20 seconds.

In other words, when calculated on the basis of data from the experimental results of Experimental Example 3, those skilled in the art can easily predict that cooling the human scalp surface to a target temperature of 0 degrees or less for a cooling time of 20 seconds or more is likely to be effective in alopecia treatment in a human. In addition, such results are found to be almost the same as the conclusion obtained when calculated on the basis of data from the experimental results of Experimental Example 2, that is, the expected results showing that cooling the human scalp surface to a target temperature of 0 degrees or less for a cooling time of 18 seconds or more would be effective in alopecia treatment in a human.

In the meantime, cooling the target area of a human to a target temperature of 5 degrees or less for a cooling time of 24 seconds or more is effective in alopecia treatment in a human. In addition, cooling the target area of a human to a target temperature of 4 degrees or less for a cooling time of 23 seconds or more is effective in alopecia treatment in a human. In addition, cooling the target area of a human to a target temperature of 3 degrees or less for a cooling time of 22 seconds or more is effective in alopecia treatment in a human. In addition, cooling the target area of a human to a target temperature of 2 degrees or less for a cooling time of 22 seconds or more is effective in alopecia treatment in a human. In addition, cooling the target area of a human to a target temperature of 1 degree or less for a cooling time of 21 seconds or more is effective in alopecia treatment in a human. In addition, cooling the target area of a human to a target temperature of −1 degrees or less for a cooling time of 20 seconds or more is effective in alopecia treatment in a human. The aforementioned combinations of target temperature and cooling time are configured to allow a cooling energy of −148,000 (W/m²) to reach human hair follicles in the same manner as described above.

In the meantime, cooling the target area of a human to a target temperature of 0 degrees or less for a cooling time of 20 seconds or more and 40 seconds or less is effective in alopecia treatment in a human. This can be seen when calculating cooling energy on the basis of the conclusion that Experimental Example 3 has demonstrated that cooling the temperature of the mouse skin surface to 0 degrees for 60 seconds is also effective in alopecia treatment. As mentioned above, the cooling energy transferred per unit area and unit time to hair follicles when cooling the mouse skin surface to 0 degrees is −4933 (W/m²), so the cooling energy transferred to the hair follicles for 60 seconds per unit area is −296,000 (W/m²). In the meantime, the cooling energy transferred per unit area and unit time to hair follicles when cooling the human scalp surface to 0 degrees is −7400 (W/m²). Therefore, in order for a cooling energy of −296,000 (W/m²) to be transferred to the human scalp, it is seen that a cooling time of 40 seconds is required.

In addition, cooling the target area of a human to a target temperature of 5 degrees or less for a cooling time of 24 seconds or more and 48 seconds or less is effective in alopecia treatment in a human. In addition, cooling the target area of a human to a target temperature of 4 degrees or less for a cooling time of 23 seconds or more and 46 seconds or less is effective in alopecia treatment in a human. In addition, cooling the target area of a human to a target temperature of 3 degrees or less for a cooling time of 22 seconds or more and 44 seconds or less is effective in alopecia treatment in a human. In addition, according to the aforementioned method, cooling the target area of a human to a target temperature of 2 degrees or less for a cooling time of 22 seconds or more and 44 seconds or less is effective in alopecia treatment in a human. In addition, according to the aforementioned method, cooling the target area of a human to a target temperature of 1 degree or less for a cooling time of 21 seconds or more and 42 seconds or less is effective in alopecia treatment in a human. In addition, according to the aforementioned method, cooling the target area of a human to a target temperature of −1 degrees or less for a cooling time of 20 seconds or more and 40 seconds or less is effective in alopecia treatment in a human. The aforementioned combinations of target temperature and cooling time are configured to allow a cooling energy of −296,000 (W/m²) to reach human hair follicles in the same manner as described above.

In the meantime, cooling the target area of a human to a target temperature of 0 degrees or less for a cooling time of 18 seconds or more and 40 seconds or less is effective in alopecia treatment in a human. In addition, cooling the target area of a human to a target temperature of 5 degrees or less for a cooling time of 21 seconds or more and 48 seconds or less is effective in alopecia treatment in a human. In addition, cooling the target area of a human to a target temperature of 4 degrees or less for a cooling time of 20 seconds or more and 46 seconds or less is effective in alopecia treatment in a human. In addition, cooling the target area of a human to a target temperature of 3 degrees or less for a cooling time of 19 seconds or more and 44 seconds or less is effective in alopecia treatment in a human. In addition, cooling the target area of a human to a target temperature of 2 degrees or less for a cooling time of 19 seconds or more and 44 seconds or less is effective in alopecia treatment in a human. In addition, cooling the target area of a human to a target temperature of 1 degree or less for a cooling time of 18 seconds or more and 42 seconds or less is effective in alopecia treatment in a human. In addition, cooling the target area of a human to a target temperature of −1 degrees or less for a cooling time of 17 seconds or more and 40 seconds or less is effective in alopecia treatment in a human.

This is because being effective in alopecia treatment is shown in both the aforementioned Experimental Example 2 (cooling to 5 degrees for 30 or 60 seconds) and Experimental Example 3 (cooling to 0 degrees for 30 or 60 seconds). Therefore, when either the target temperature and cooling time calculated according to Experimental Example 2 or the target temperature and cooling time calculated according to Experimental Example 3 are satisfied, it is likely to be effective in alopecia treatment in a human.

In the meantime, cooling the target area of a human to a target temperature of 0 degrees or less for a cooling time of 20 seconds or more and 36 seconds or less is effective in alopecia treatment in a human. In addition, cooling the target area of a human to a target temperature of 5 degrees or less for a cooling time of 24 seconds or more and 42 seconds or less is effective in alopecia treatment in a human. In addition, cooling the target area of a human to a target temperature of 4 degrees or less for a cooling time of 23 seconds or more and 40 seconds or less is effective in alopecia treatment in a human. In addition, cooling the target area of a human to a target temperature of 3 degrees or less for a cooling time of 22 seconds or more and 38 seconds or less is effective in alopecia treatment in a human. In addition, cooling the target area of a human to a target temperature of 2 degrees or less for a cooling time of 22 seconds or more and 38 seconds or less is effective in alopecia treatment in a human. In addition, cooling the target area of a human to a target temperature of 1 degree or less for a cooling time of 21 seconds or more and 36 seconds or less is effective in alopecia treatment in a human. In addition, cooling the target area of a human to a target temperature of- 1 degrees or less for a cooling time of 20 seconds or more and 34 seconds or less is effective in alopecia treatment in a human. This is because being effective in alopecia treatment is shown in both Experimental Examples 2 and 3. Therefore, when both the target temperature and cooling time calculated according to Experimental Example 2 and the target temperature and cooling time calculated according to Experimental Example 3 are satisfied, the alopecia treatment effect in a human is certainly achievable.

In the meantime, according to the aforementioned method, the combination of the target temperature to which the target area of a human is cooled and the cooling time being effective in alopecia treatment in a human, depending on the target temperature, may be calculated. For example, according to the aforementioned method, when the target temperature is less than-1 degrees, such as −2 degrees, −3 degrees, −4 degrees, −5 degrees, −6 degrees, −7 degrees, −8 degrees, or −9 degrees, the cooling time capable of treating alopecia may be found. In addition, when the target temperature is more than 5 degrees, such as 6 degrees, 7 degrees, 8 degrees, 9 degrees, or 10 degrees, the cooling time capable of treating alopecia may be found. In addition, the cooling time capable of treating alopecia in a human may be calculated at target temperatures other than the aforementioned target temperatures and combined.

In addition, in terms of the cooling time derived as mentioned above, 18 seconds or more may be interpreted as 17 seconds or more. In addition, 20 seconds or more may be interpreted as 19 seconds or more. In addition, 36 seconds or less may be interpreted as 37 seconds or less. For example, 40 seconds or less may be interpreted as 41 seconds or less, or 42 seconds or less. This is because the derived cooling time is predicted to be equally effective in alopecia treatment within a ±5% range under the doctrine of equivalents.

In addition, in terms of the cooling time derived as mentioned above, 18 seconds or more may be interpreted as 16 seconds or more, or 17 seconds or more. In addition, 20 seconds or more may be interpreted as 18 seconds or more, or 19 seconds or more. In addition, 36 seconds or less may be interpreted as 37 seconds or less, 38 seconds or less, or 39 seconds or less. In addition, 40 seconds or less may be interpreted as 41 seconds or less, 42 seconds or less, 43 seconds or less, or 44 seconds or less. This is because the derived cooling time is predicted to be equally effective in alopecia treatment within a ±10% range under the doctrine of equivalents.

In addition, in terms of the cooling time derived as mentioned above, 18 seconds or more may be interpreted as 15 seconds or more, 16 seconds or more, or 17 seconds or more. In addition, 20 seconds or more may be interpreted as 17 seconds or more, 18 seconds or more, or 19 seconds or more. In addition, 36 seconds or less may be interpreted as 37 seconds or less, 38 seconds or less, 39 seconds or less, 40 seconds or less, or 41 seconds or less. In addition, 40 seconds or less may be interpreted as 41 seconds or less, 42 seconds or less, 43 seconds or less, 44 seconds or less, 45 seconds or less, or 46 seconds or less. This is because the derived cooling time is predicted to be equally effective in alopecia treatment within a ±15% range under the doctrine of equivalents.

In addition, in terms of the cooling time derived as mentioned above, 18 seconds or more may be interpreted as 14 seconds or more, 15 seconds or more, 16 seconds or more, or 17 seconds or more. In addition, 20 seconds or more may be interpreted as 16 seconds or more, 17 seconds or more, 18 seconds or more, or 19 seconds or more. In addition, 36 seconds or less may be interpreted as 37 seconds or less, 38 seconds or less, 39 seconds or less, 40 seconds or less, 41 seconds or less, 42 seconds or less, or 43 seconds or less. In addition, 40 seconds or less may be interpreted as 41 seconds or less, 42 seconds or less, 43 seconds or less, 44 seconds or less, 45 seconds or less, 46 seconds or less, 47 seconds or less, or 48 seconds or less. This is because the derived cooling time is predicted to be equally effective in alopecia treatment within a ±20% range under the doctrine of equivalents.

As mentioned above, each of the cooling times capable of treating alopecia in a human is predicted to be equally effective in alopecia treatment in a human within the respective ±5%, ±10%, ±15%, and ±20% ranges under the doctrine of equivalents.

### Spray-Type Cryotherapy Apparatus Used to Treat Alopecia in Human

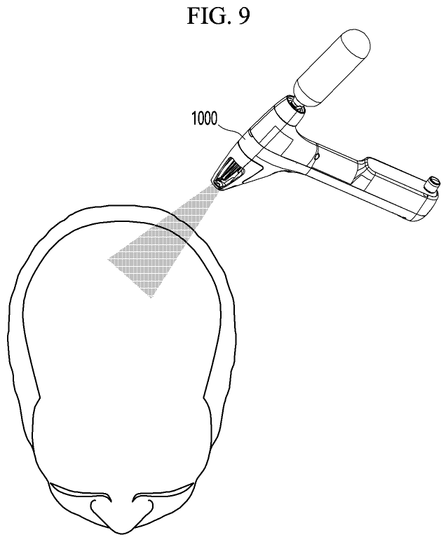

As can be seen in FIG. 9, an alopecia treatment method for a human may also be performed in the same manner as the alopecia treatment method for an animal by spraying the coolant on the target area using the spray-type cryotherapy apparatus 1000. Thus, the spray-type cryotherapy apparatus 1000 used to treat alopecia in a human may be the same as the spray-type cryotherapy apparatus 1000 used to treat alopecia in an animal. Therefore, the description of the spray-type cryotherapy apparatus 1000 used to treat alopecia in a human may be the same as that in the aforementioned [Spray-Type Cryotherapy Apparatus 1000 Used to Treat Alopecia in Animal]. In addition, the spray-type cryotherapy apparatus 1000 described with reference to FIGS. 2 to 5 can also be used to treat alopecia in a human, so a detailed description thereof is to be omitted.

### Alopecia Treatment Method for Human

The alopecia treatment method for a human may also be performed in the same manner as that described in [Alopecia Treatment Method for Animal]. In other words, the alopecia treatment method for an animal, as described with reference to FIGS. 6 to 8, is equally applicable to the alopecia treatment method for a human. However, in terms of applying [Alopecia Treatment Method for Animal] to [Alopecia Treatment Method for Human], the target area that the nozzle of the spray-type cryotherapy apparatus 1000 faces is a part of the human scalp, where alopecia occurs. In other words, in the alopecia treatment method for a human, the target area that the nozzle of the spray-type cryotherapy apparatus 1000 faces should not be interpreted as a part of the animal skin. Furthermore, in terms of applying [Alopecia Treatment Method for Animal] to [Alopecia Treatment Method for Human], a target temperature and cooling time that are input, set, or stored in the spray-type cryotherapy apparatus 1000 may be any one of the combinations of target temperature and cooling time described in [Target Temperature and Cooling Time Capable of Treating Alopecia in Human].

### Contact-Type Cryotherapy Apparatus Used to Treat Alopecia in Animal or Human

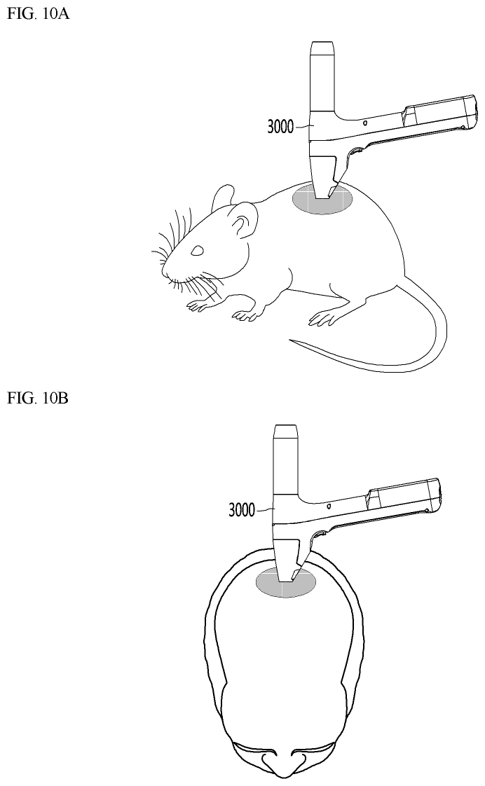

In the meantime, as can be seen in FIGS. 10A and 10B, a contact-type cryotherapy apparatus 3000, as well as the spray-type cryotherapy apparatus, can also be used to treat alopecia in an animal or human using cryotherapy. Accordingly, the contact-type cryotherapy apparatus 3000 used to treat alopecia in an animal or human is to be described. Hereinafter, the term “target area” used in describing the method of treating alopecia using the contact-type cryotherapy apparatus 3000 is a part of the human scalp or a part of the animal skin surface and refers to an area in contact with a cooling medium or cooling tip of the contact-type cryotherapy apparatus.

In the meantime, the contact-type cryotherapy apparatus 3000 used for alopecia treatment in a human or animal should not be limitedly interpreted as the contact-type cryotherapy apparatus 3000, to be described later. Any device capable of cooling the target area by contacting the target area with a part of the contact-type cryotherapy apparatus 3000 should be interpreted as the contact-type cryotherapy apparatus for alopecia treatment described in the present disclosure.

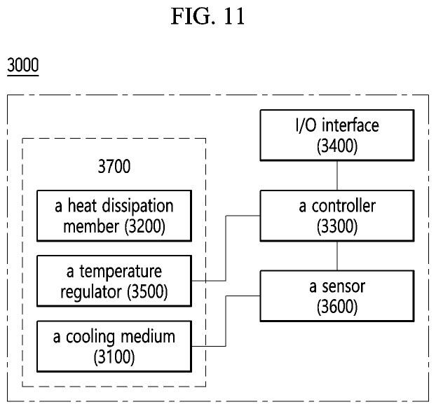

FIG. 11 illustrates configurations of the contact-type cryotherapy apparatus 3000 capable of being used for alopecia treatment, according to one embodiment of the present disclosure. Referring to FIG. 11, the contact-type cryotherapy apparatus 3000 may include a cooling module 3700, a sensor 3600, an I/O interface 3400, and a controller 3300. The cooling module 3700 may generate cooling energy to provide the generated cooling energy to the target area. The cooling module 3700 may include a cooling medium 3100, a temperature regulator 3500, and a heat dissipation member 3200. The cooling module 3700 may adjust the temperature of the cooling medium 3100 using the temperature regulator 3500 and may allow the temperature of the target area to reach a target temperature and be maintained through the cooling medium 3100. For example, the cooling medium 3100 may be in direct contact with the target area to provide cooling energy to the target area or may provide cooling energy to the target area through a cooling tip (not shown) thermally coupled to the cooling medium 3100, thereby allowing the temperature of the target area to reach the target temperature and be maintained.

The heat dissipation member 3200 may release heat generated by the operation of the cooling module 3700 to the outside. For example, the heat dissipation member 3200 may receive heat generated as the temperature regulator 3500 generates cooling energy and may release the generated heat to the outside of the contact-type cryotherapy apparatus 3000. The heat dissipation member 3200 may be thermally coupled to the temperature regulator 3500 to receive thermal energy. The heat dissipation member 3200 may be made of a thermally conductive material, such as metal, to release heat efficiently.

The temperature regulator 3500 may adjust the temperature of the cooling medium 3100. For example, the temperature regulator 3500 may receive a control signal from the controller 3300, to be described later, to provide cooling energy or thermal energy to the cooling medium 3100. In addition, the temperature regulator 3500 may, for example, be thermally coupled to the cooling medium 3100 to provide cooling energy or thermal energy to the cooling medium 3100. Furthermore, the temperature regulator 3500 may be implemented in various forms. For example, the temperature regulator 3500 may include a thermoelement configured to be supplied with current and operate on the basis of the Peltier effect, the thermoelement having one surface absorbing heat and the other surface releasing heat depending on the direction of the supplied current. For another example, the temperature regulator 3500 may include a device or element based on thermodynamic cycles such as a vapor compression refrigeration cycle or Stirling cooler, or a device or element based on the Joule-Thomson effect using expanding gas. For another example, the temperature regulator 3500 may generate or provide cooling energy using coolants such as carbon dioxide or liquid nitrogen.

The sensor 3600 may detect the physical properties of the contact-type cryotherapy apparatus 3000. For example, the sensor 3600 may be directly or indirectly coupled to the cooling medium 3100 to measure the current, voltage, or temperature of the cooling medium 3100. In addition, the sensor 3600 may provide the detected physical properties of the contact-type cryotherapy apparatus 3000 to the controller 3300. For example, the sensor 3600 may provide real-time temperature values, voltage/current values, and the like of the cooling medium 3100 to the controller 3300. Furthermore, the sensor 3600 may include a touch sensor or pressure sensor to determine whether the contact-type cryotherapy apparatus 3000 has been contacted to the target area.

The I/O interface 3400 has the same function as the I/O interface 1400 of the aforementioned spray-type cryotherapy apparatus 1000. For example, at least one target temperature and at least one cooling time may be input into the I/O interface 3400 and transmitted to the controller 3300. A detailed description of the I/O interface 3400 is substituted with the description of the I/O interface 1400 of the spray-type cryotherapy apparatus 1000.

The controller 3300 may control the overall operation of the contact-type cryotherapy apparatus 3000.

The controller 3300 may control the temperature regulator 3500 to adjust the temperature of the cooling medium 3100, and the cooling medium 3100 may provide cooling energy to the target area to cool the target. Hereinafter, a process of performing a cooling function is to be described in detail.

The controller 3300 may provide cooling energy to the cooling medium 3100 by controlling the temperature regulator 3500 so that the temperature of the cooling medium 3100 may be adjusted to a temperature that enables the temperature of the target area to become the target temperature. For example, the controller 3300 may control the output of the temperature regulator 3500 on the basis of the temperature information obtained from the sensor 3600 to increase, decrease, or maintain the temperature of the cooling medium 3100, thereby adjusting the temperature of the cooling medium 3100. Accordingly, the temperature of the target area may be allowed to reach the target temperature and be maintained at the target temperature for a cooling time. In the meantime, when the cooling time has elapsed, the controller 3300 may provide an alert to a user or may stop controlling the output of the temperature regulator 3500, thus stopping cooling of the target area.

### Alopecia Treatment Method Using Contact-Type Cryotherapy Apparatus 3000

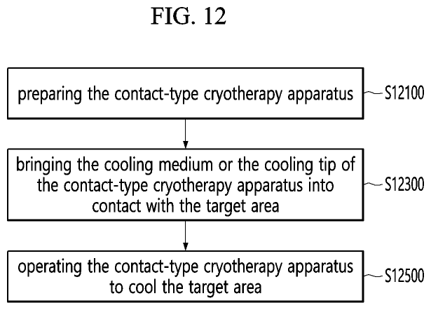

With reference to FIGS. 12 to 14, the method of treating alopecia in a human or animal using the aforementioned contact-type cryotherapy apparatus 3000 or contact-type cryotherapy apparatuses having different structures is to be described. The alopecia treatment method for a human or animal using the contact-type cryotherapy apparatus 3000 may be similar to [Alopecia Treatment Method for Animal] and [Alopecia Treatment Method for Human] described on the basis of FIGS. 6 to 8. Specifically, S12100, S12300, and S12500 in FIG. 12 may correspond to S6100, S6300, and S6500 in FIG. 6, respectively. However, in the description based on FIG. 6, the “spray-type cryotherapy apparatus ” should be interpreted as “contact-type cryotherapy apparatus ”. Furthermore, “positioning the nozzle of the spray-type cryotherapy apparatus to face the target area” in the description of S6300 should be understood as “contacting the cooling medium 3100 or the cooling tip (not shown) of the contact-type cryotherapy apparatus with the target area” in the description of S12300.

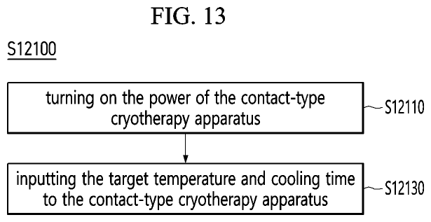

In the meantime, S12110 and S12130 in FIG. 13 are also performed in the same manner as S6130 and S6150 in FIG. 7, except that the “contact-type cryotherapy apparatus ” is used rather than the “spray-type cryotherapy apparatus ”, so a detailed description thereof is to be omitted.

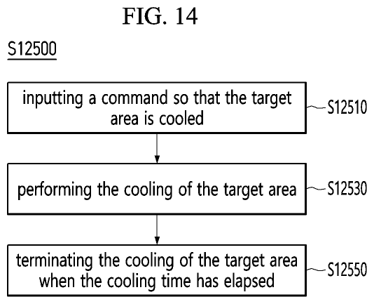

Furthermore, S12510 to S12550 in FIG. 14 are also similar to S6510 to S6530 in FIG. 8, so a detailed description thereof is to be omitted. However, the “spray-type cryotherapy apparatus ” should be interpreted as the “contact-type cryotherapy apparatus ” in FIG. 14, and “spraying the coolant on the target area” should be interpreted as “adjusting the temperature of the cooling medium to adjust the temperature of the target area to the target temperature”.

In the meantime, when an alarm sounds after the cooling time has elapsed, a user using the contact-type cryotherapy apparatus 3000 may separate the contact-type cryotherapy apparatus 3000 from the target area so that the cooling energy of the cooling medium 3100 is prevented from being transferred to the target area. Alternatively, when an alarm sounds, a user may terminate cooling of the target area by turning the power of the contact-type cryotherapy apparatus 3000 off or stopping the operation of the contact-type cryotherapy apparatus 3000. Alternatively, when the cooling time has elapsed, the contact-type cryotherapy apparatus 3000 may stop cooling of the target area.

In the meantime, even with the use of the contact-type cryotherapy apparatus 3000 rather than the spray-type cryotherapy apparatus 1000, cooling performed on the target area, such as the human scalp or the animal skin, by selecting the target temperature and cooling time described in [Target Temperature and Cooling Time Capable of Treating Alopecia in Animal] or [Target Temperature and Cooling Time Capable of Treating Alopecia in Human], as mentioned above, is likely to be effective in alopecia treatment equally or similarly to cooling performed using the spray-type cryotherapy apparatus 1000.

### Method of Treating Alopecia Through Split Cooling

[Alopecia Treatment Method for Animal], [Alopecia Treatment Method for Human], and [Alopecia Treatment Method Using Contact-Type Cryotherapy Apparatus 3000] above have introduced the method of treating alopecia as follows: selecting the target temperature and the cooling time, spraying the coolant or adjusting the temperature of the cooling medium in contact with the target area to allow the temperature of the target area to reach the target temperature, and continuously spraying the coolant uninterruptedly for the cooling time or controlling the temperature of the cooling medium in contact with the target area.

In the meantime, when the target temperature is 0 degrees or less and the cooling time is 20 seconds or more, alopecia in a human is treatable when performing cooling for a split cooling time of 10 seconds or more, stopping cooling for a cooling pause time of 4 seconds or less, and performing cooling again for a split cooling time of 10 seconds or more. This will be seen through Experimental Example 4, to be described later.

In other words, as long as the total cooling time for which the target area is maintained at the target temperature is the same as the aforementioned cooling time, intermittent cooling performed on the target area by dividing the total cooling time into two or more split cooling times is effective in alopecia treatment. In particular, as long as the cooling pause time, during which the target area is not cooled between split cooling times, is short enough that the cooling effect achieved during the preceding split cooling time is not lost, cooling performed over two or more split cooling times is as effective in alopecia treatment as cooling continuously performed on the target area uninterruptedly for the cooling time.

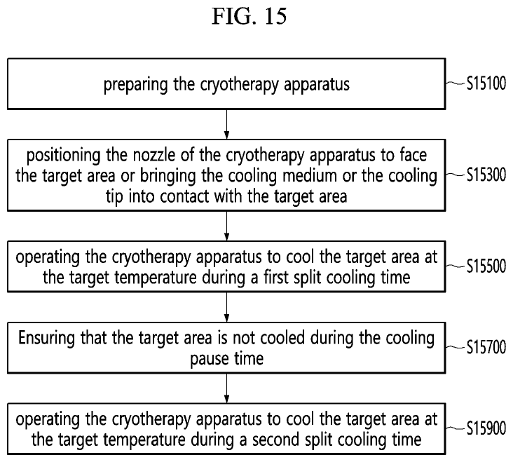

FIG. 15 is a view for describing the method of treating alopecia by setting the total of a plurality of split cooling times the same as the cooling time, as mentioned above. In the following description, the cryotherapy apparatus may refer to the spray-type cryotherapy apparatus 1000 or the contact-type cryotherapy apparatus 3000. S15100 is the same as S6100 in FIG. 6, the preparation of the spray-type cryotherapy apparatus according to FIG. 7, S12100 in FIG. 12, and the preparation of the contact-type cryotherapy apparatus according to FIG. 13, so a detailed description thereof is to be omitted. Similarly, S15300 is the same as S6300 in FIG. 6 and S12300 in FIG. 12, so a detailed description thereof is to be omitted. In the meantime, a user may operate the cryotherapy apparatus so that the temperature of the target area reaches the target temperature and the temperature of the target area is maintained at the target temperature for a first split cooling time (S15500). Then, when the first split cooling time has elapsed, the user keeps the target area from being cooled by the cryotherapy apparatus for a cooling pause time (S15700). In this case, the operation of the user keeping the target area from being cooled for the cooling pause time may be the same as the action performed by the user in S6530 in FIG. 8 when the cooling time has elapsed or the action performed by the user in S12550 in FIG. 14 when the cooling time has elapsed. When the first split cooling time has elapsed, the cryotherapy apparatus sounds an alarm, so the user may be aware of the initiation of the cooling pause time. In the meantime, when the cooling pause time has elapsed, the user may operate the cryotherapy apparatus so that the temperature of the target area reaches the target temperature and the temperature of the target area is maintained at the target temperature for a second split cooling time (S15900). In this case, the cryotherapy apparatus may notify the user through an alarm that the cooling pause time has elapsed. In the meantime, the operations in S15500 and S15700 are the same as S6500 in FIG. 6 and the operation of FIG. 8 or similar to S12500 in FIG. 12 and the operation in FIG. 14, so a detailed description thereof is to be omitted. However, in the description above, “cooling time” should be understood as the “first split cooling time” or “second split cooling time”. In the meantime, the cooling time may be divided into three or more split cooling times, so that cooling may be performed at each split cooling time, and there may be a cooling pause time between adjacent split cooling times. When the cooling time is divided into three or more split cooling times, S15700 and S15900 may be repeatedly performed until the total cooling time for which cooling is performed during all split cooling times is equal to the cooling time selected above. For example, when there are three split cooling times, S15700 and S15900 may be repeatedly performed once more after S15900. Alternatively, when there are four split cooling times, S15700 and S15900 may be repeatedly performed twice more after S15900. Alternatively, when there are five split cooling times, S15700 and S15900 may be repeatedly performed three more times after S15900.

In the meantime, for example, the cooling pause time may be short enough that the cooling effect achieved when cooling the target area for the preceding split cooling time is not lost. For example, the cooling pause time may be shorter than at least one of the split cooling times. For example, the cooling pause time may be shorter than the preceding split cooling time. For example, the cooling pause time may be shorter than the shortest of the split cooling times. For example, the cooling pause time may be shorter than the longest of the split cooling times. In the meantime, since there are three or more split cooling times, each cooling pause time may be the same or different in duration when there are two or more cooling pause times.

In the meantime, the total of the split cooling times may be the same as the cooling time selected above. For example, each of the split cooling times may be substantially the same. For example, two or more of the split cooling times may have the same first duration, and the remaining split cooling times may have the same second duration. For example, all the split cooling times may be different in duration. For example, a part of split cooling times may have a first duration, a part of split cooling times may have a second duration, and the remaining split cooling times may have a third duration. For example, a part of split cooling times may have a first duration, a part of split cooling times may have a second duration, a part of split cooling times may have a third duration, and the remaining split cooling times may have a fourth duration. In this case, the first to fourth durations may all be different.

For example, when the target temperature is 0 degrees or less and the cooling time is 20 seconds or more, cooling may be performed for a split cooling time of 10 seconds or more, stopped for a cooling pause time of 4 seconds or less, and performed again for a split cooling time of 10 seconds or more. For example, when the target temperature is 0 degrees or less and the cooling time is 20 seconds or more, the operation in which cooling is performed for a split cooling time of 5 seconds or more, stopped for a cooling pause time of 2 seconds or less, performed again for a split cooling time of 5 seconds or more, and stopped again for a cooling pause time of 2 seconds or less may be repeatedly performed twice. For example, when the target temperature is 0 degrees or less and the cooling time is 20 seconds or more, the following operation may be performed: spraying the coolant for a split cooling time of 7 seconds or more, stopping the cooling for a cooling pause time of 4 seconds or less, spraying the coolant for a split cooling time of 5 seconds or more, stopping the cooling for a cooling pause time of 3 seconds or less, spraying the coolant for a split cooling time of 6 seconds or more, stopping the cooling for a cooling pause time of 2 seconds or less, and spraying the coolant for a split cooling time of 2 seconds or more. In this case, the split cooling times that occur when the operation is repeatedly performed four times may all be the same in duration but may be different, as mentioned above.

In the meantime, to perform split cooling, the user may input the target temperature into the cryotherapy apparatus while inputting the determined number of split cooling times and cooling pause times. For example, when the user inputs 0 degrees or less as the target temperature, 10 seconds twice as the split cooling time, and 4 seconds as the cooling pause time, the cryotherapy apparatus may provide an alert when the target area reaches 0 degrees and 10 seconds have elapsed thereafter, or may stop the coolant spraying or cooling operation. Then, the cryotherapy apparatus may provide an alert again or may perform the coolant spraying or cooling operation after 4 seconds have elapsed Then, the cryotherapy apparatus may provide an alert again or may stop the coolant spraying or cooling operation when the target area reaches 0 degrees and 10 seconds have elapsed thereafter. To this end, the user may set the cryotherapy apparatus to operate in a split cooling mode. In addition, the cryotherapy apparatus set to operate in a split cooling mode may load any one of the pre-stored combinations of target temperature and cooling time to perform split cooling according to the loaded combination. In this case, the user may set the cryotherapy apparatus and select the combination of target temperature and cooling time to be loaded.

In the meantime, whether split cooling is effective in alopecia treatment, as mentioned above, is to be described in detail through Experimental Example 4, to be described later.

### Treatment Frequency and Duration in Alopecia Treatment Method

When one cryotherapy session is defined as cooling that allows a target area, which is a part of the animal skin or the human scalp, to reach a selected target temperature and the temperature of the target area to be maintained at the target temperature for a selected cooling time, it has been explained above that even a single session of such cryotherapy could be effective in alopecia treatment. However, when the aforementioned cryotherapy is repeatedly performed multiple times, the aforementioned alopecia treatment effect may be further enhanced.

For example, when such cryotherapy is performed once or more, or twice or more in a single alopecia treatment session, the alopecia treatment effect may be further enhanced. For example, when the single alopecia treatment session is performed once or more, or twice or more per week, the alopecia treatment effect may be further enhanced. For example, when the single alopecia treatment session is performed once or more, or twice or more per week for at least four weeks, at least eight weeks, or at least twelve weeks, the alopecia treatment effect may be further enhanced.

In the meantime, this is to be described through Experimental Example 4, to be described later.

### Method of Administering Aforementioned Alopecia Treatment Method for Prevention of Alopecia

When the alopecia treatment method based on the target temperature and cooling time described in this specification is performed on an area where alopecia has not occurred, alopecia may be prevented from occurring. This is because the degree of activation of the alopecia treatment-related genetic factors when performing the aforementioned alopecia treatment method has been confirmed to be comparable to or greater than the degree of activation of the alopecia treatment-related genetic factors when minoxidil is applied on the target area, as mentioned above.

In the meantime, minoxidil is a representative therapeutic for alopecia but has already been used widely to prevent alopecia. This is because the mechanism by which minoxidil is applied on an alopecic area to treat alopecia is manifested in the same manner even when minoxidil is applied on an area where alopecia has not occurred. In other words, the application of minoxidil on an area where alopecia has not occurred may help normally maintain the anagen and telogen phases of hair growth by maintaining the condition of hair follicles and may supply nutrients continuously to hair by preventing blood vessels from narrowing or disappearing, thus preventing alopecia from occurring. Accordingly, when alopecia can be prevented through minoxidil, the alopecia prevention effect can be obtained through the aforementioned alopecia treatment method.

Thus, the aforementioned alopecia treatment method should not be interpreted as being used only for alopecia treatment, but should be understood as also being performed to prevent alopecia.

### Alopecia Conditions to Which Aforementioned Alopecia Treatment Method Can Be Applied

In the meantime, the alopecia treatment method, according to the present disclosure, can be used for any alopecia regardless of the causes thereof.

Alopecia may be classified into androgenetic alopecia in men, androgenetic alopecia in women, alopecia areata, and telogen effluvium. Androgenetic alopecia in men and androgenetic alopecia in women are types of alopecia that occur due to genetic factors. Expression of such genetic factors in men is called androgenetic alopecia in men, while expression of such genetic factors in women is called androgenetic alopecia in women. In addition, alopecia areata, a type of autoimmune disease, results from damage to hair follicles or hair due to reasons such as inflammatory cells attacking hair follicles. Moreover, telogen effluvium is a disease that occurs when the hair growth cycle enters the telogen phase excessively fast. While minoxidil has been used to treat or prevent all of the aforementioned types of alopecia, the effect thereof has been recognized. However, as mentioned above, it has already been described that the degree of activation of the alopecia treatment-related genetic factors when using the alopecia treatment method, according to the present disclosure, is comparable to or greater than that observed when using minoxidil. Thus, the alopecia treatment method described in the present disclosure can be used for any alopecia, regardless of the causes thereof. In the meantime, Experimental Example 4, to be described later, demonstrates that the aforementioned alopecia treatment method is effective for androgenetic alopecia in men.

### Experimental Example 1

Experimental Example 1 is a nonclinical experiment conducted on mice, which is designed to determine whether the anagen phase of hair growth was induced at all target temperatures and cooling times when performing cooling with varying target temperatures and varying cooling times.

### [1] Experimental Preparation

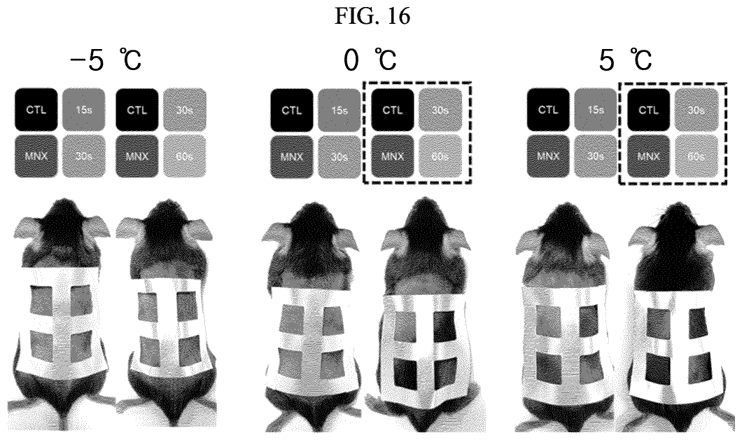

FIG. 16 shows the experimental preparation to perform Experimental Example 1. As can be seen in FIG. 16, six mice were prepared in Experimental Example 1. In this case, six mice were 7-week-old C 57BL/6 nonclinical laboratory mice. After shaving the dorsal side of each mouse, the hair-free dorsal part was divided into four areas.

To perform cooling on two out of the four areas divided on the dorsal side of each mouse, Target Cool, manufactured by Recens Medical Inc., was used. Target Cool, a spray-type cryotherapy apparatus configured to spray a coolant, is a product that enables precise temperature cooling control so that after cooling a target area to a target temperature set by a user, the temperature of the target area is maintained at the target temperature for a set cooling time. In the meantime, carbon dioxide served as the coolant used for cooling.

### [2] Experimental Method

The four areas on the dorsal side of a first mouse of the six mice were treated as follows. A first area, serving as a control group, was left untreated. A second area was where minoxidil was applied at a concentration of 2%. A third area was cooled to a target temperature of −5 degrees by spraying the coolant on the target area for 15 seconds. A fourth area was cooled to a target temperature of −5 degrees by spraying the coolant on the target area for 30 seconds.

The four areas on the dorsal side of a second mouse of the six mice were treated as follows. A first area, serving as a control group, was left untreated. A second area was where minoxidil was applied at a concentration of 2%. A third area was cooled to a target temperature of −5 degrees by spraying the coolant on the target area for 30 seconds. A fourth area was cooled to a target temperature of −5 degrees by spraying the coolant on the target area for 60 seconds.

In the meantime, the four areas on the dorsal side of a third mouse were treated as follows. A first area, serving as a control group, was left untreated. A second area was where minoxidil was applied at a concentration of 2%. A third area was cooled to a target temperature of 0 degrees by spraying the coolant on the target area for 15 seconds. A fourth area was cooled to a target temperature of 0 degrees by spraying the coolant on the target area for 30 seconds.

In addition, the four areas on the dorsal side of a fourth mouse were treated as follows. A first area, serving as a control group, was left untreated. A second area was where minoxidil was applied at a concentration of 2%. A third area was cooled to a target temperature of 0 degrees by spraying the coolant on the target area for 30 seconds. A fourth area was cooled to a target temperature of 0 degrees by spraying the coolant on the target area for 60 seconds.

In addition, the four areas on the dorsal side of a fifth mouse were treated as follows. A first area, serving as a control group, was left untreated. A second area was where minoxidil was applied at a concentration of 2%. A third area was cooled to a target temperature of 5 degrees by spraying the coolant on the target area for 15 seconds. A fourth area was cooled to a target temperature of 5 degrees by spraying the coolant on the target area for 30 seconds.

In addition, the four areas on the dorsal side of a sixth mouse were treated as follows. A first area, serving as a control group, was left untreated. A second area was where minoxidil was applied at a concentration of 2%. A third area was cooled to a target temperature of 5 degrees by spraying the coolant on the target area for 30 seconds. A fourth area was cooled to a target temperature of 5 degrees by spraying the coolant on the target area for 60 seconds.

In the meantime, cooling was performed eight times on the third and fourth areas of each mouse in Experimental Example 1. Specifically, cooling was performed on the third and fourth areas of each mouse twice per week for 4 weeks.

### [3] Observation Method of Experimental Results

After performing cooling for 4 weeks, the experimenter collected skin tissues from each of the first to fourth areas on the dorsal side of each of the six mice, followed by performing 4′,6-diamidino-2-phenylindole (DAPI) staining and versican staining on the collected skin tissues to stain the versican protein expressed in the dermal papillae of hair follicles and cell nuclei. Then, whether the anagen phase of hair growth was induced was observed using a fluorescence microscope. In addition, the experimenter collected other skin tissues separately from each of the first to fourth areas on the dorsal side of each of the six mice, followed by performing hematoxylin and eosin (H&E) staining on the other skin tissues separately collected to stain the cell nuclei, cytoplasm, and the like. Then, the structure of the dorsal skin tissue of the mice and whether the anagen phase of hair growth was induced were observed using a fluorescence microscope. In the meantime, when collecting skin tissues from the dorsal side of each of the six mice, the six mice were anesthetized. In addition, after collecting skin tissues, the six mice were euthanized.

### [4] Experimental Results

As a result of the experiment, in the case of the first and second mice, where cooling was performed to −5 degrees, the induction of the anagen phase of hair growth was not observed in the respective third and fourth areas. In addition, in the case of the third mouse, where cooling was performed to a target temperature of 0 degrees for a cooling time of 15 seconds, the induction of the anagen phase of hair growth was not observed in the third area. Furthermore, in the case of the fifth mouse, where cooling was performed to a target temperature of 5 degrees for a cooling time of 15 seconds, the induction of the anagen phase of hair growth was not observed in the third area.

On the other hand, the induction of the anagen phase of hair growth was observed in the fourth area of the third mouse and the third area of the fourth mouse, where cooling was performed to a target temperature of 0 degrees for a cooling time of 30 seconds. In addition, the induction of the anagen phase of hair growth was observed in the fourth area of the fifth mouse and the third and fourth areas of the sixth mouse, where cooling was performed to a target temperature of 5 degrees for a cooling time of 30 or 60 seconds.

### [5] Analysis of Experimental Results

The experimental results showed that no alopecia treatment effect was observed when cooled to −5 degrees, regardless of the cooling time, and no alopecia treatment effect was observed when the cooling time was set to 15 seconds, regardless of the target temperature. On the other hand, it was seen that the anagen phase of hair growth was induced when cooling the skin to 0 degrees for 30 seconds and to 5 degrees for 30 or 60 seconds. Accordingly, the applicant was able to find out that simply cooling the skin surface did not necessarily result in the alopecia treatment effect and that an appropriate target temperature and cooling time for alopecia treatment should be selected to achieve the alopecia treatment effect.

### Experimental Example 2

In Experimental Example 1, the potential of the alopecia treatment effect was observed when cooling the mouse skin surface to 5 degrees for 30 and 60 seconds. On this basis, Experimental Example 2 was designed to find out the degree of induction of the anagen phase of hair growth and the degree of activation of the alopecia treatment-related genetic factors of FGF21, VEGF, CIPR, RBM3, and PGC1a when cooling the mouse skin surface to 5 degrees for 30 and 60 seconds.

### [1] Experimental Preparation

In Experimental Example 2, the experimenter prepared three 7-week-old C 57BL/6 nonclinical laboratory mice. After shaving the dorsal side of each mouse, the hair-free dorsal part was divided into four areas. To perform cooling on two out of the four areas of each mouse, the aforementioned Target Cool, manufactured by Recens Medical Inc., was prepared, and carbon dioxide served as a coolant.

### [2] Experimental Method

The experimenter treated each of the four areas of the three mice as follows. A first area, serving as a control group, was left untreated by the experimenter. The experimenter applied minoxidil at a concentration of 2% on a second area. The experimenter cooled a third area to a target temperature of 5 degrees by spraying the coolant on the target area for 30 seconds. The experimenter cooled a fourth area to a target temperature of 5 degrees by spraying the coolant on the target area for 60 seconds.

In the meantime, cooling was performed eight times on the third and fourth areas of each mouse in Experimental Example 2. Specifically, cooling was performed on the third and fourth areas of each mouse twice per week for 4 weeks.

### [3] Observation Method of Experimental Results

The experimenter observed the induction of the anagen phase of hair growth by the same observation method as in Experimental Example 1. In addition, to measure the mRNA expression levels of the alopecia treatment-related genetic factors, the experimenter collected other skin tissues separately from each of the first to fourth areas on the dorsal side of each of the three mice, followed by extracting RNA from the other skin tissues separately collected. Then, mRNA was selectively isolated from the extracted RNA, and the isolated mRNA was reversely transcribed into cDNA, followed by performing a quantitative polymerase chain reaction (qPCR) to measure the relative expression level of each alopecia treatment-related genetic factor. In this case, the fold change value was used as a measure of the relative difference in expression.

### [4] Experimental Results and Their Interpretation

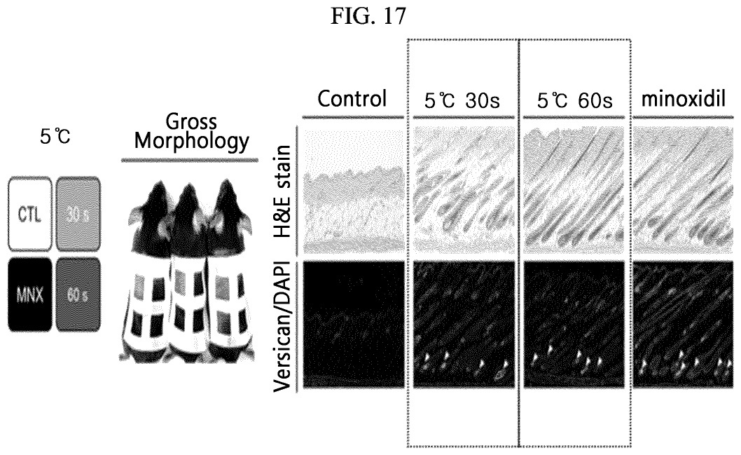

FIG. 17 shows the degree of induction of the anagen phase of hair growth, according to Experimental Example 2. In FIG. 17, the upper image shows H&E staining results, and the lower image shows the DAPI and versican staining results. In the meantime, white triangle marks in the DAPI and versican staining results indicate the dermal papillae stained with versican. As can be seen in FIG. 17, compared to the control group, the dermal papillae were activated in the areas cooled to a target temperature of 5 degrees for 30 seconds and cooled to a target temperature of 5 degrees for 60 seconds, and hair grew in the hair follicles, showing that the anagen phase of hair growth was definitely induced. In addition, it was confirmed that the degree of induction of the anagen phase of hair growth in the areas cooled to a target temperature of 5 degrees for 30 seconds and cooled to a target temperature of 5 degrees for 60 seconds was comparable to, or even greater than, that in the area where minoxidil was applied.

In the meantime, it was seen that the anagen phase of hair growth was induced more in the area cooled for 60 seconds than in the area cooled for 30 seconds. This shows that the longer the cooling time, the better the alopecia treatment effect.

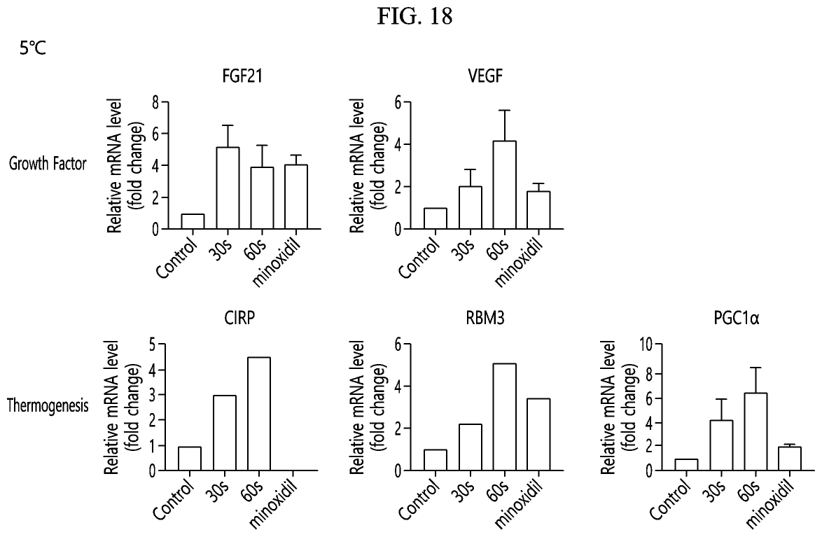

FIG. 18 shows the mRNA expression levels of the five alopecia treatment-related genetic factors, according to Experimental Example 2. Referring to FIG. 18, it was seen that the mRNA expression levels of all five alopecia treatment-related genetic factors in the area cooled to a target temperature of 5 degrees for 30 seconds were higher than those in the control group and those in the area where minoxidil was applied.

In the meantime, the mRNA expression levels of all five alopecia treatment-related genetic factors in the area cooled to a target temperature of 5 degrees for 60 seconds were higher than those in the control group. In addition, it was seen that the expression levels of all four alopecia treatment-related genetic factors, except for FGF21, when cooled to a target temperature of 5 degrees for 60 seconds, were higher than those in the area where minoxidil was applied. In the meantime, it was confirmed that the expression level of FGF21 when cooled to a target temperature of 5 degrees for 60 seconds was slightly lower than, but almost comparable to, the expression level of FGF21 in the area where minoxidil was applied. In consideration of the above, it was confirmed that the alopecia treatment effect of cooling to a target temperature of 5 degrees for 60 seconds would be comparable to or better than that of minoxidil. In addition, in comparison of when cooled to a target temperature of 5 degrees for 60 seconds with when cooled to a target temperature of 5 degrees for 30 seconds, it was seen that the expression levels of all four genetic factors, except for FGF21, were significantly high in 60-second cooling. Although the expression level of FGF 21 was slightly low in 60-second cooling, the difference from 30-second cooling was insignificant. When referring to the activation of such alopecia treatment-related genetic factors, it was confirmed that the alopecia treatment effect was better when cooled to 5 degrees for 60 seconds than when cooled to 5 degrees for 30 seconds. In other words, at the same temperature of 5 degrees, the expression levels of the alopecia treatment-related genetic factors were overall higher when cooled for 60 seconds than when cooled for 30 seconds. For this reason, it was confirmed that 60-second cooling is better than 30-second cooling in the alopecia treatment effect. In consideration of the above, it is confirmed that, when cooled for 30 seconds or more, the longer the cooling time, the better the alopecia treatment effect. This interpretation may be derived because, as mentioned above, the longer the cooling time, the greater the amount of cooling energy transferred to the hair follicles, resulting in higher expression levels of the alopecia treatment-related genetic factors. Accordingly, it is confirmed through Experimental Example 2 that cooling the mouse skin to a target temperature of 5 degrees for 30 seconds or more is effective in alopecia treatment.

### Experimental Example 3

In Experimental Example 1, the experimenter observed the potential of the alopecia treatment effect when cooling the mouse skin surface to 0 degrees for 30 seconds. On this basis, Experimental Example 3 was designed to find out the degree of induction of the anagen phase of hair growth and the degree of activation of the alopecia treatment-related genetic factors of FGF21, VEGF, CIPR, RBM3, and PGC1a when cooling the mouse skin surface to 5 degrees for 30 and 60 seconds.

### [1] Experimental Preparation

Experimental Example 3 was prepared in the same manner as Experimental Example 2.

### [2] Experimental Method

An experiment was conducted in the same manner as in Experimental Example 2, except for changing the target temperature of the third and fourth areas from 5 degrees to 0 degrees.

### [3] Observation Method of Experimental Results

The experimental results were observed in the same manner as in Experimental Example 2.

### [4] Experimental Results and Their Interpretation

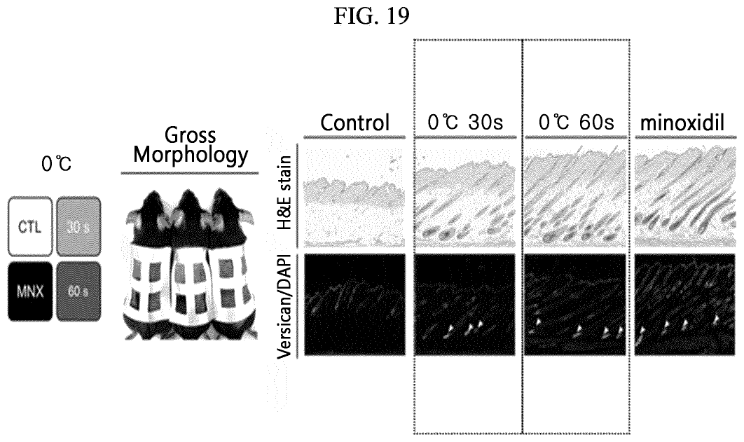

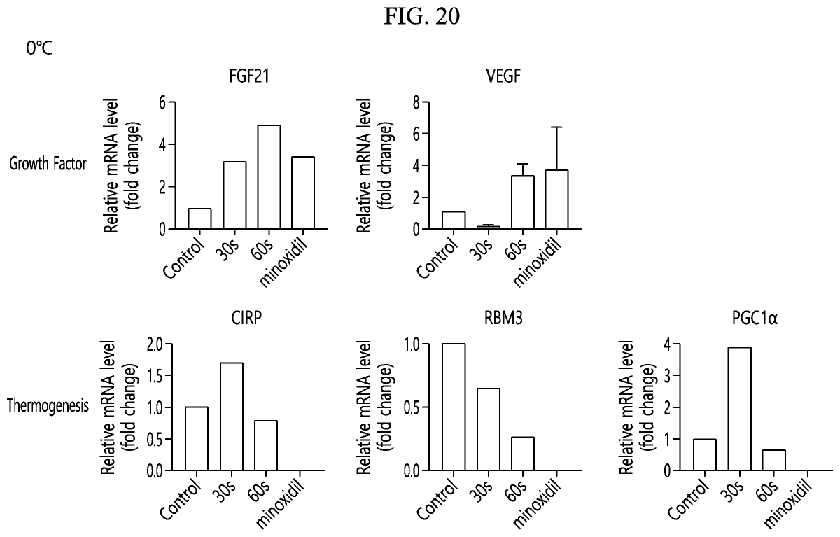

FIG. 19 shows the degree of induction of the anagen phase of hair growth, as in FIG. 17, and FIG. 20 shows the degree of mRNA activation of the alopecia treatment-related genetic factors, as in FIG. 18. Referring to FIGS. 19 and 20, the experimenter confirmed the induction of the anagen phase of hair growth and activation of the alopecia treatment-related genetic factors in the areas cooled to a target temperature of 0 degrees for 30 seconds and to a target temperature of 0 degrees for 60 seconds when compared to the control group. In addition, it was seen that the alopecia treatment effect was comparable to or better than that of minoxidil. Therefore, it was seen through Experimental Example 3 that cooling the mouse skin to a target temperature of 0 degrees for 30 seconds or more was effective in alopecia treatment.

In the meantime, in Experimental Example 2, it was seen that cooling to a target temperature of 5 degrees for 30 seconds or more was effective in alopecia treatment. In addition, in Experimental Example 3, it was seen that cooling to a target temperature of 0 degrees for 30 seconds or more was effective in alopecia treatment. Therefore, taking into account all the experimental results of Experimental Examples 2 and 3 together, it is seen that cooling to a target temperature of 5 degrees or less for 30 seconds or more is effective in alopecia treatment. For example, it is seen that cooling to a target temperature of 0 degrees or more and 5 degrees or less for 30 seconds or more is effective in alopecia treatment. For example, it is seen that cooling to a target temperature of 0 degrees or more and 5 degrees or less for 30 seconds or more and 60 seconds or less is effective in alopecia treatment.

### Experimental Example 4

Experimental Example 4 is an experiment designed to find out whether performing split cooling on a human is effective in alopecia treatment.

### [1] Experimental Design

Five test subjects (human, male) with androgenetic alopecia in men were recruited. The above-described Target Cool, manufactured by Recens Medical Inc., served as a cryotherapy apparatus configured to perform cooling on the five test subjects, and carbon dioxide served as a coolant.

### [2] Experimental Method

The experimenter measured hair thickness and density in the target area, a part of the scalp of each test subject, before performing alopecia treatment according to the present disclosure. In this case, the area of the target area was 121.174 mm\hat{ }2.

In addition, the experimenter continuously sprayed the coolant on the target area for 10 seconds after the target area reached −1 degrees, stopped the coolant spraying on the target area, and sprayed the coolant on the target area again 3 to 4 seconds thereafter, thus continuously spraying the coolant on the target area for 10 seconds after the target area reached −1 degrees. In other words, in Experimental Example 4, a total cooling time of 20 seconds was divided into two split cooling times of 10 seconds each to perform split cooling, and a cooling pause time of 3 to 4 seconds was set between the two split cooling times. In addition, spraying, as mentioned above, is set as one cryotherapy session, and two cryotherapy sessions were performed on each test subject per day. In addition, the two cryotherapy sessions mentioned above were performed twice per week.

As mentioned above, two cryotherapy sessions were performed each time twice per week to measure hair thickness and density in the target area at 4-week intervals, thereby observing the changes in hair thickness and density over a total of 12 weeks.

In the meantime, the hair thickness and density were measured using the Follicope program. Specifically, a photograph of the target area was taken and then input into the Follicope program. In addition, for hair density measurement, the number of hairs per follicle in the target area was counted, and the hair follicles were labeled according to the number of hairs. Then, the number of hairs growing in the target area according to the corresponding label was all added to measure the density and the rate of increase in density in the target area.

Furthermore, for hair thickness measurement, the thickness of each hair in the target area was measured. In this case, the thickness of each hair was measured manually by clicking on both ends of each hair with a computer mouse. Then, the average value of the hair thicknesses measured by the aforementioned method was recorded as the hair thickness of the test subject.

### [3] Experimental Results and Their Interpretation

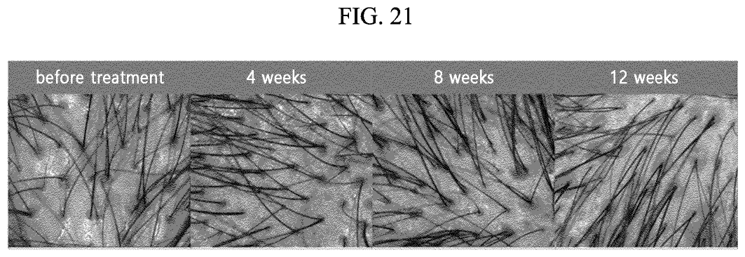

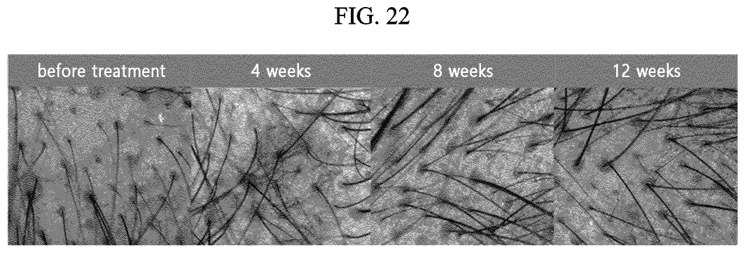

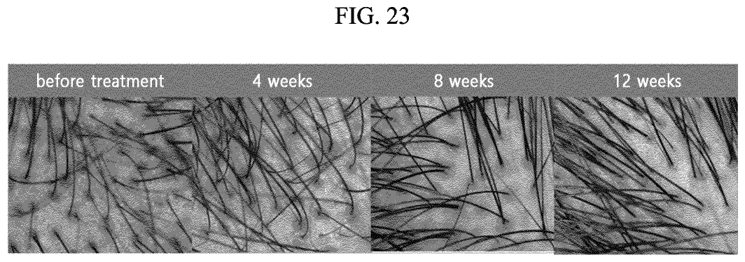

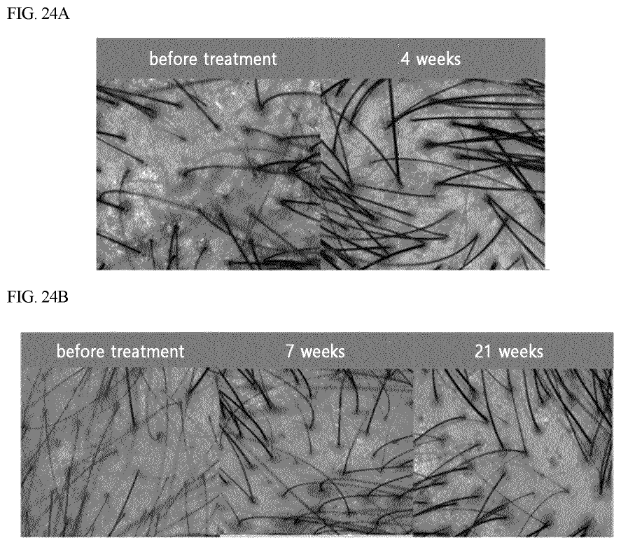

FIGS. 21 to 23 show photographs of the scalp (that is, the target area) of first, second, and third test subjects taken before the treatment and taken at 4-week intervals after the start of the treatment. FIG. 24A shows a photograph of the scalp (that is, the target area) of a fourth test subject taken before the treatment and taken 4 weeks after the start of the treatment. FIG. 24B shows a photograph of the scalp (that is, the target area) of a fifth test subject taken before the treatment and taken 7 weeks and 21 weeks after the start of the treatment.

FIGS. 21 to 24B show that the number and density of hairs in the target area increase.

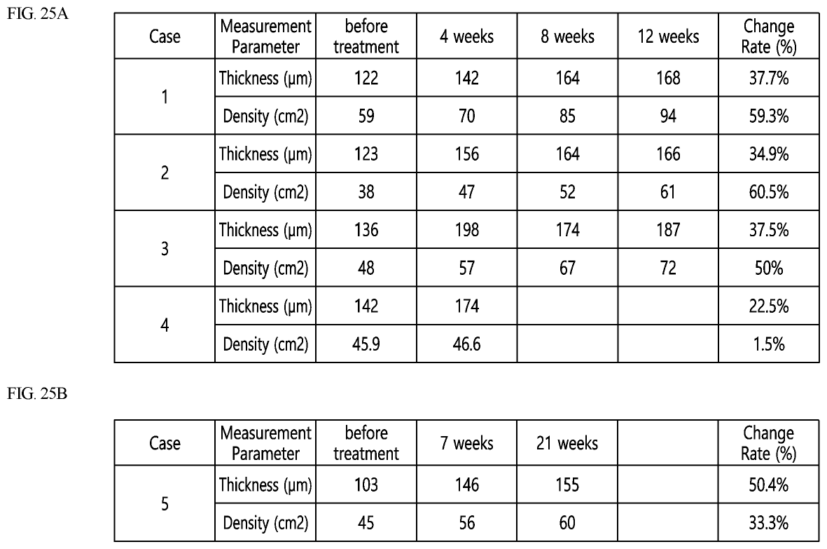

FIG. 25A shows the observation of the hair thickness and density of the first to fourth test subjects, the results thereof measured before the treatment and measured at 4-week intervals. In addition, FIG. 25B shows the observation of the hair thickness and density of the fifth test subject, the results thereof measured before the treatment and measured after 7 weeks and 21 weeks after the start of the treatment. As in the results in FIG. 24, FIG. 25A also shows that the hair thickness and density increase in just 4 weeks after the start of the cryotherapy and that the hair thickness and density further increase at Weeks 8 and 12. In particular, when comparing the hair thickness and density at Week 12 with the hair thickness and density before the treatment, it is shown that the hair thickness increases by about 35%, and the hair density increases by about 50% to 60%.

In addition, referring to FIG. 25B, the hair thickness and density further increase at Weeks 7 and 21. When comparing the hair thickness and density at Week 21 with the hair thickness and density before the treatment, it is shown that the hair thickness increases by about 50%, and the hair density increases by about 33%.

Androgenetic alopecia in men presents two symptoms, one of which is the gradual thinning of hair shafts, and the other is the eventual shedding of the thinned hairs from hair follicles. However, both test subjects with androgenetic alopecia in men experienced an increase in hair thickness. This means that hair thinned due to androgenetic alopecia in men has become thicker again, and that the symptom of androgenetic alopecia in men, where the hair thickness is gradually thinned, has been prevented and treated. In addition, both test subjects with androgenetic alopecia in men experienced an increase in hair density. This means that hair has grown again from the hair follicles where hair has already been shed, and that the symptom of androgenetic alopecia in men, where hairs are shed from the hair follicles, has been prevented and treated. Thus, it is seen that all symptoms of androgenetic alopecia in men are improved with the aforementioned cryotherapy.

In the meantime, Experimental Example 4 shows that spraying the coolant for a total of 20 seconds by performing split cooling to a target temperature of −1 degrees for 10 seconds each is effective in alopecia treatment. However, the temperature difference between −1 degrees and 0 degrees is extremely marginal. When calculated according to the aforementioned Fourier's law of heat conduction, the difference between the cooling energy transferred per unit area and unit time to the hair follicles in the human scalp at −1 degrees and the cooling energy transferred per unit area and unit time to the hair follicles in the human scalp at 0 degrees is only 200 (W/m²). However, considering that the total amount of cooling energy transferred per unit area and unit time to the hair follicles in the human scalp at 0 degrees is 7400 (W/m²), a difference of 200 (W/m²) is small. In addition, as described in the present disclosure, referring to the conversion results for the application of the amount of cooling energy calculated through Experimental Examples 2 and 3 to a human, the cooling time at 0 degrees and −1 degrees are either the same, or the difference thereof is calculated as less than 1 second. Therefore, through Experimental Example 4, where spraying the coolant on the target area to a target temperature of −1 degrees continuously for 20 seconds or more is proven effective in alopecia treatment, it is seen that cooling the target area to a target temperature of 0 degrees or less for a total of 20 seconds or more is likely to be effective in alopecia treatment.

In addition, the experimental conditions (that is, the target temperature and cooling time) of Experimental Example 4 closely correspond to the target temperature and cooling time expected to be effective in alopecia treatment in a human on the basis of Experimental Examples 2 and 3. In addition, it was shown that the alopecia treatment effects in Experimental Examples 2 and 3 resulted from the induction of the anagen phase of hair growth and sufficient activation of the five alopecia treatment-related genetic factors mentioned above. This leads to an expectation that in Experimental Example 4, the induction of the anagen phase of hair growth and sufficient activation of the five alopecia treatment-related genetic factors mentioned above result from the mechanism by which androgenetic alopecia in men has been treated. In the meantime, Experimental Example 4 shows that when a practitioner cools the scalp to a target temperature of 0 degrees or less for a total of 20 seconds or more, a meaningful alopecia treatment effect can be obtained despite intermittently spraying the coolant according to the aforementioned split cooling method, without continuously spraying the coolant. In other words, Experimental Examples 2, 3, and 4 show that it is effective when a practitioner sprays the coolant on the target area to a target temperature of 0 degrees or less for a total of 20 seconds or more through split cooling, as well as confirming that it is effective for alopecia even when a practitioner sprays the coolant on the target area to a target temperature of 0 degrees or less continuously for 20 seconds or more.

### Prophetic Experimental Example 1

Prophetic Experimental Example 1 is an experiment designed to demonstrate that, even when cooling the mouse skin surface using a contact-type cryotherapy apparatus under the same target temperature and cooling time as in the aforementioned Experimental Example 2, the anagen phase of hair growth is induced, and the alopecia treatment-related genetic factors of FGF21, VEGF, CIPR, RBM3, and PGC1a are activated to a degree comparable to or greater than that observed with the application of minoxidil.

### [1] Experimental Preparation

Prophetic Experimental Example 1 was prepared in the same manner as Experimental Example 2, except for using a contact-type cryotherapy apparatus, including all configurations described in FIG. 11, as a device configured to cool two out of the four areas divided on the dorsal side of each mouse. For example, OcuCool, manufactured by Recens Medical Inc., may be used. No coolant was involved because the contact-type cryotherapy apparatus was used.

### [2] Experimental Method

Prophetic Experimental Example 1 was performed in the same manner as Experimental Example 2, except for contacting the contact-type cryotherapy apparatus with the target area. In other words, except for the coolant spraying, the target temperature and cooling time of the third and fourth areas were the same as those in Experimental Example 2, and the number of cooling cycles was also the same.

### [3] Observation Method of Experimental Results

The experimental results were observed in the same manner as in Experimental Example 2.

### [4] Determination Method of Experimental Results

When the degree of induction of the anagen phase of hair growth in the third and fourth areas of each mouse is comparable to or greater than that in the second area, where minoxidil was applied, it is likely to be determined that the alopecia treatment effect is observed in the third and fourth areas. In addition, when the degree of induction of the anagen phase of hair growth is greater in 60-second cooling than in 30-second cooling at a target temperature of 5 degrees, it is likely to be determined that the alopecia treatment effect is better in 60-second cooling than in 30-second cooling. Furthermore, when the cooling time is longer than 30 seconds or more, it is likely to be determined that the longer the cooling time, the better the alopecia treatment effect.

In addition, when the mRNA expression levels of all alopecia treatment-related genetic factors in the third and fourth areas of each mouse are higher than those in the control group as well as those in the area where minoxidil is applied, or when the mRNA expression levels of at least three or more of the alopecia treatment-related genetic factors in the third and fourth areas of each mouse are at least comparable to those in the area where minoxidil is applied, this means that the expression levels of the majority of the genetic factors is higher than those observed with the application of minoxidil. Therefore, it is likely to be determined that the alopecia treatment effect is observed in the third and fourth areas. In addition, where there are at least three or more genetic factors the mRNA expression levels of which are higher in 60-second cooling than in 30-second cooling at a target temperature of 5 degrees, it is likely to be determined that the alopecia treatment effect is better in 60-second cooling than in 30-second cooling. Furthermore, when the cooling time is longer than 30 seconds or more, it is likely to be determined that the longer the cooling time, the better the alopecia treatment effect.

### Prophetic Experimental Example 2

Prophetic Experimental Example 2 is an experiment designed to demonstrate that, even when cooling the mouse skin surface using a contact-type cryotherapy apparatus under the same target temperature and cooling time as in the aforementioned Experimental Example 3, the anagen phase of hair growth is induced, and the alopecia treatment-related genetic factors of FGF21, VEGF, CIPR, RBM3, and PGC1a are activated to a degree comparable to or greater than that observed with the application of minoxidil.

### [1] Experimental Preparation

Prophetic Experimental Example 2 was prepared in the same manner as Experimental Example 3, except for using a contact-type cryotherapy apparatus, including all configurations described in FIG. 11, as a device configured to cool two out of the four areas divided on the dorsal side of each mouse. For example, OcuCool, manufactured by Recens Medical Inc., may be used. No coolant was involved because the contact-type cryotherapy apparatus was used.

### [2] Experimental Method

Prophetic Experimental Example 2 was performed in the same manner as Experimental Example 3, except for contacting the contact-type cryotherapy apparatus with the target area. In other words, except for the coolant spraying, the target temperature and cooling time of the third and fourth areas were the same as those in Experimental Example 3, and the number of cooling cycles was also the same.

### [3] Observation Method of Experimental Results

The experimental results were observed in the same manner as in Experimental Example 3.

### [4] Determination Method of Experimental Results

Whether the alopecia treatment effect was observed was determined on the basis of the same criteria as in “Determination Method of Experimental Results” in Prophetic Experimental Example 1.

### Prophetic Experimental Example 3

Prophetic Experimental Example 3 is an experiment designed to demonstrate that, even when using a contact-type cryotherapy apparatus on a human, cooling the scalp under the same target temperature, split cooling time, and cooling pause time as in Experimental Example 4 is effective in alopecia treatment.

### [1] Experimental Design

Prophetic Experimental Example 3 was prepared in the same manner as Experimental Example 4. However, a contact-type cryotherapy apparatus, including all configurations described in FIG. 11, was used as a cryotherapy apparatus for cooling. For example, OcuCool, manufactured by Recens Medical Inc., may be used. No coolant was involved because the contact-type cryotherapy apparatus was used. In the meantime, the experiment was conducted by recruiting at least five test subjects aged 20 or older with androgenetic alopecia in men.

### [2] Experimental Method

Prophetic Experimental Example 3 was performed in the same manner as in Experimental Example 4, except for setting the target temperature to 0 degrees. The cooling time, cooling method, and cryotherapy frequency and cycle were the same.

### [3] Determination Method of Experimental Results

As in Experimental Example 4, when the hair thickness and density increase by at least 30% or more, it is likely to be determined that the symptoms of androgenetic alopecia in men are improved, indicating that the cryotherapy is effective in treating and preventing androgenetic alopecia in men.

Although the present disclosure has been described with reference to specific embodiments and drawings, it will be appreciated that various modifications and changes can be made from the present disclosure by those skilled in the art. For example, suitable results may be achieved if the described techniques are performed in a different order and/or if components in a described system, architecture, device, or circuit are combined in a different manner and/or replaced or supplemented by other components or their equivalents.

Therefore, other implementations, embodiments, and equivalents are within the scope of the following claims.

What is claimed is:

1. A method of treating or preventing androgenetic alopecia of a human using a cryotherapy apparatus in a subject, the method comprising:
providing the cryotherapy apparatus, wherein the cryotherapy apparatus comprises a nozzle, a valve, a coolant storage connector coupled to a coolant storage, and at least one pipe fluidically connecting the nozzle, the value, and the coolant storage connector;
positioning the nozzle of the cryotherapy apparatus to face a target area of the subject, the target area is a part of a human scalp;
operating the cryotherapy apparatus to spray a coolant to the target area such that a temperature of the target area reaches to a target temperature and a cooling time of the target area is equal to or longer than a total cooling time period, wherein the target temperature is equal to or less than 5 degrees Celsius and the total cooling time period is equal to or longer than 17 seconds; and
controlling the cryotherapy apparatus to output an alert or stop spraying the coolant after the target area is cooled to the target temperature for the cooling time.

2. The method of claim 1,
wherein the operating the cryotherapy apparatus comprises:
operating the cryotherapy apparatus to spray the coolant continuously from a cooling starting time for the total cooling time period to a cooling terminating time for the total cooling time period, and
whereby the target area is cooled to the target temperature and maintained its temperature for the total cooling time period.

3. The method of claim 1,
wherein the operating the cryotherapy apparatus comprises:
operating the cryotherapy apparatus to spray the coolant intermittently from a cooling starting time for the total cooling time period to a cooling terminating time for the total cooling time period,
whereby the target area is cooled to the target temperature and maintained its temperature for a first cooling time period, and then the target area is cooled again to the target temperature and maintained its temperature for a second cooling time period,
wherein a sum of the first cooling time period and the second cooling time period is same to the total cooling time period.

4. The method of claim 3, wherein a cooling pause time period between the first time and the second time is shorter than at least one of the first cooling time period and the second cooling time period.

5. The method of claim 3, wherein the first cooling time period is substantially identical with the second cooling time period.

6. The method of claim 3, wherein a cooling pause time period is predetermined such that cooling effect of the target area due to the first cooling time period does not disappear during the cooling pause time period.

7. The method of claim 3, wherein each of the first cooling time period and the second cooling time period is equal to or longer than 10 seconds.

8. The method of claim 7, wherein a cooling pause time period is equal to or shorter than 4 seconds.

9. The method of claim 1, wherein the total cooling time period is equal to or shorter than 40 seconds, 42 seconds, 44 seconds, 46 seconds, 48 seconds, or 50 seconds.

10. The method of claim 1, wherein the total cooling time period is equal to or shorter than 60 seconds, 63 seconds, 66 seconds, 69 seconds, 72 seconds, or 75 seconds.

11. The method of claim 1, the method activates at least one of FGF21, VEGF, CIRP, RBM3 and PGC 1a of the human.

12. The method of claim 1, the method induces an anagen phase of hair growth of the human scalp.

13. The method of claim 1, wherein the coolant is liquified carbon dioxide (CO2).

14. The method of claim 1, wherein the method is repeatedly performed for at least 4 weeks, at least 8 weeks or at least 12 weeks.

15. The method of claim 12, wherein the method is repeatedly performed at least once per week or at least twice per week.

16. The method of claim 12, wherein the method is defined as a cycle, at least two cycles are repeatedly performed at least once per week or at least twice per week.

17. The method of claim 1, wherein the cryotherapy apparatus further comprises a temperature sensor detecting the temperature of the target area and a temperature regulator,
wherein at operating the cryotherapy apparatus to spray the coolant,
the temperature regulator regulate a temperature of the coolant to reaching and maintaining the temperature of the target area to the target temperature based on the detected temperature of the target area and the target temperature.

18. A cryotherapy apparatus of treating or preventing androgenetic alopecia of a human, the cryotherapy apparatus comprising:
a valve configured to allow a flow of a coolant which is provided from a coolant storage;
a temperature regulator configured to regulate a temperature of the coolant;
a nozzle configured to spray the coolant;
a temperature sensor configured to measure a temperature of a target area which is a part of a human scalp; and
a controller configured to the valve, the temperature sensor and the temperature regulator,
wherein the controller is further configured to:
set a target temperature to cool and maintain the target area to the set target temperature,
receive, from the temperature sensor, the measured temperature of the target area,
control the temperature regulator to regulate a temperature of the coolant based on the target temperature and the measured temperature of the target area,
control the valve to spray the coolant through the nozzle such that the temperature of the target area reaches to the target temperature and a cooling time of the target area is equal to or longer than a total cooling time period, wherein the target temperature is equal to or less than 5 degrees Celsius and the total cooling time period is equal to or longer than 17 seconds, and
output an alert or control the valve to stop spraying the coolant after the target area is cooled to the target temperature for the cooling time.

19. A cryotherapy apparatus of treating or preventing alopecia of a human, the cryotherapy apparatus comprising:
a valve configured to allow a flow of a coolant which is provided from a coolant storage;
a nozzle configured to spray the coolant; and
a controller configured to the valve,
wherein the controller is further configured to:
set a target temperature to cool and maintain a target area, which is a part of a human scalp, to the set target temperature,
control the valve to spray the coolant through the nozzle such that a temperature of the target area reaches to the target temperature and a cooling time of the target area is equal to or longer than a total cooling time period, wherein the target temperature is equal to or less than 5 degrees Celsius and the total cooling time period is equal to or longer than 17 seconds, and
output an alert or control the valve to stop spraying the coolant after the target area is cooled to the target temperature for the cooling time.
